(19) United States
(12) Patent Application Publication
(10) Pub. No.: US 2013/0109586 A1
(43) Pub. Date: May 2, 2013

(54) GENERATION OF ANTIBODIES TO AN EPITOPE OF INTEREST THAT CONTAINS A PHOSPHOMIMETIC AMINO ACID

(71) Applicant: KaloBios Pharmaceuticals, Inc., South San Francisco, CA (US)

(72) Inventors: Kenneth Luehrsen, South San Francisco, CA (US); Geoffrey T. Yarranton, South San Francisco, CA (US); Christina Yi, South San Francisco, CA (US); Lu Bai, South San Francisco, CA (US)

(73) Assignee: KaloBios Pharmaceuticals, Inc., South San Francisco, CA (US)

(21) Appl. No.: 13/644,353

(22) Filed: Oct. 4, 2012

Related U.S. Application Data
(60) Provisional application No. 61/543,746, filed on Oct. 5, 2011.

(51) Int. Cl. G01N 33/68

(52) U.S. Cl. CPC G01N 33/6854
USPC 506/9; 530/387.1

(57) ABSTRACT

The invention provides methods of obtaining antibodies to an epitope of interest based on an anti-phosphoamino acid-focused library.

### CROSS-REFERENCE TO RELATED APPLICATIONS

This application claims priority benefit of U.S. provisional application No. 61/543,746, filed Oct. 5, 2011, which application is herein incorporated by reference for all purposes.

### BACKGROUND OF THE INVENTION

A major challenge in antibody technology is the generation of monoclonal antibodies that selectively bind to a pre-chosen epitope on an antigen of interest. The reason for this is usually due to the fact that proteins often have dominant epitopes (i.e., epitopes that are efficiently recognized by the host immune system) and/or epitopes for which the host immune system is tolerized (treated as a self-antigen). The repertoire of antibodies that bind to an antigen can therefore be quite restricted. A number of strategies are being developed to address this issue including: new adjuvants, chimeric peptides and DNA vaccination (Grunewald et al., Proc. Natl. Acad Sci USA 105:11276-11280, 2008). However, these approaches do not direct the immune response to a defined site on the protein or peptide of interest. Further, these approaches are not performed in vitro, and thus are constrained by the endogenous immune system of the host organism. In vitro methods of de novo antibody generation, e.g., phage display technology, also rely on the use of antibody libraries made from naturally occurring V-region sequences. Such libraries tend to be biased due to the in vivo tolerance mechanisms of the host organism from which the V-region libraries were made. The present method overcomes these limitations by providing in vivo and in vitro methods to generate an antibody to a desired epitope.

### BRIEF SUMMARY OF THE INVENTION

The invention relates to methods of obtaining an antibody targeted to a defined epitope within a peptide or protein antigen and libraries and reagents for performing such methods. In some aspects, the invention involves modifying an epitope of interest by replacing a glutamic acid or an aspartic acid residue within the desired epitope with a phosphoamino acid (e.g., phosphoserine or phosphothreonine). High affinity antibodies can be selected that bind the phosphoamino acid and additional amino acids present in the epitope. This aspect of the invention takes advantage of the physical similarities of naturally occurring phosphoamino acids, such as phosphoserine or phosphothreonine, with the amino acids glutamic acid and aspartic acid. Glutamic acid and aspartic acid share similar carbon spacing and negative charge (at neutral pH) with the phosphoamino acids phosphoserine and phosphothreonine and are thus phosphomimetics. The invention is thus based, in part on the discovery that high affinity antibodies directed against an epitope containing a phosphoamino acid will also bind an epitope in which the phosphoamino acid is replaced with a naturally occurring amino acid, e.g., aspartic acid or glutamic acid, that is a phosphomimetic.

Peptide and protein antigens that contain phosphoamino acids can be prepared by chemical synthesis, by in vitro labeling with a protein kinase or by in vivo methods. Phosphoamino acid-containing antigens are known to provoke a strong immunological response in mice and rabbits. Additionally, diverse libraries of antibodies that bind phosphoamino acids (independent of any surrounding amino acids) can be prepared by recombinant DNA methodology. Thus, a peptide or protein antigen labeled with a phosphoamino acid can be used to select (either in vivo or in vitro) an antibody that: 1) binds the epitope that contains the phosphoamino acid (i.e. phosphoserine or phosphothreonine), 2) also binds the epitope that contains a phosphomimetic amino acid (e.g., glutamic acid or aspartic acid) and 3) does not bind the epitope when the phosphoamino acid is replaced by the non-phosphorylated form of the amino acid (i.e. serine or threonine).

The invention can be used to generate monoclonal antibodies to protein epitopes identified as therapeutic targets. Additionally, the invention can be used to design more effective immunization strategies that target therapeutic epitopes on foreign proteins or that break immunological tolerance to self-antigens.

In one aspect, the invention relates to a method of obtaining an antibody to an epitope of interest containing an aspartic acid or glutamic acid, the method comprising: (a) obtaining an anti-phosphoamino acid-focused antibody library generated from a reference antibody, wherein the members of the library retain at least one minimal essential binding specificity determinant of a CDR from the reference antibody V_{H} or V_{L}; (b) screening the anti-phosphoamino acid-focused antibody library with a phosphoamino acid-containing epitope, wherein the epitope of interest has a phosphoamino acid substituted for the glutamic acid or for the aspartic acid; (c) identifying members of the anti-phosphoamino acid-focused antibody library that exhibit better binding to the phosphoamino acid-containing epitope in comparison to the reference antibody, which defines a sublibrary of the anti-phosphoamino acid-focused library; (d) screening the sublibrary of step (c) with the epitope of interest that has the glutamic acid or aspartic acid; and (e) selecting an antibody from screening step (d) that binds to the epitope of interest. In some embodiments, step (b) further comprises screening the anti-phosphoamino acid-focused antibody library with a phosphoamino acid comparator to identify members that exhibit better binding to the phosphamino acid-containing epitope than to the phosphamino acid comparator.

In another aspect, the invention relates to a method of obtaining an antibody to an epitope of interest containing an aspartic acid or glutamic acid, the method comprising: (a) obtaining an anti-phosphoamino acid-focused antibody library generated from a reference antibody, wherein the members of the library retain at least one minimal essential binding specificity determinant of a CDR from the reference antibody V_{H} or V_{L}; (b) screening the anti-phosphoamino acid-focused antibody library with a phosphoamino acid-containing epitope, wherein the epitope of interest has a phosphoamino acid substituted for the glutamic acid or for the aspartic acid; (c) identifying members of the anti-phosphoamino acid-focused antibody library that exhibit better binding to the phosphoamino acid-containing epitope in comparison to the reference antibody, which defines a sublibrary of the anti-phosphoamino acid-focused library; (d) selecting one of the V regions of an antibody chain of an antibody identified in step (c) and exchanging a cassette of the selected V region with a library of corresponding cassettes to provide a library of engineered V regions, wherein the selected V region retains at least one minimal essential binding specificity determinant of a CDR from the antibody identified in step (c); (e) pairing the V region library of step (d) with a complementary V region, or a diverse library of complementary V regions, wherein the complementary V region or the diverse library of complementary V regions comprise an MEBSD from the reference antibody; (f) screening the library of step (e) with the epitope of interest that has the glutamic acid or aspartic acid; and (g) selecting an antibody that binds to the epitope wherein the antibody comprises an engineered V region. In some embodiments, step (b) further comprises screening the anti-phosphoamino acid-focused antibody library with a phosphoamino acid comparator to identify members that exhibit better binding to the phosphamino acid-containing epitope than to the phosphamino acid comparator. In some embodiments, the diverse library of complementary V regions in step (e) comprises members that have at least one exchange cassette exchanged with corresponding cassettes that have diverse sequences. In some embodiments, the selected V region of step (d) is a heavy chain V region. In other embodiments, the selected V region of step (d) is a light chain V region. In some embodiments, the cassette that is exchanged in step (d) is a CDR3-FR4 cassette.

In an additional aspect, the invention relates to a method of obtaining an antibody to an epitope of interest containing an aspartic acid or glutamic acid, the method comprising: (a) obtaining an anti-phosphoamino acid-focused antibody library generated from a reference antibody, wherein the members of the library retain at least one minimal essential binding specificity determinant of a CDR from the reference antibody V_{H} or V_{L}; (b) screening the anti-phosphoamino acid-focused antibody library with a phosphoamino acid-containing epitope, wherein the epitope of interest has a phosphoamino acid substituted for the glutamic acid or for the aspartic acid; (c) identifying members of the anti-phosphoamino acid-focused antibody library that exhibit better binding to the phosphoamino acid-containing epitope in comparison to the reference antibody, which defines a sublibrary of the anti-phosphoamino acid-focused library; (d) selecting one of the V regions of an antibody chain of an antibody identified in step (c) and pairing the V region with a diverse library of complementary V regions to form a library of antibodies; (e) screening the library of step (d) with the epitope of interest that has the glutamic acid or aspartic acid; and (f) selecting an antibody that binds to the epitope of interest. In some embodiments, step (b) further comprises screening the anti-phosphoamino acid-focused antibody library with a phosphoamino acid comparator to identify members that exhibit better binding to the phosphamino acid-containing epitope than to the phosphamino acid comparator. In some embodiments, the V region selected in step (d) is a V_{H} region. In other embodiments, the V region selected in step (d) is a V_{L} region.

In various embodiments of the invention, the members of the anti-phosphoamino acid-focused library retain a minimal essential binding specificity determinant from the reference antibody CDR3 V_{H} or V_{L} region. In some embodiments, the members of the anti-phosphoamino acid-focused library retain a minimal essential binding specificity determinant from the reference antibody heavy chain CDR3 and a minimal essential binding specificity determinant from the reference antibody light chain CDR3. In some embodiments, the members of the anti-phosphoamino acid-focused library retain the reference antibody heavy chain CDR3 and the reference antibody light chain CDR3.

In some embodiments of the invention, the phosphoamino acid is phosphoserine or phosphothreonine, which can be substituted for either an aspartic acid or a glutamic acid present in an epitope of interest. In some embodiments, the aspartic acid or glutamic acid is naturally occurring in the sequence of the epitope of interest.

In some embodiments, the phosphoamino acid-containing epitope employed in the invention is a peptide of from 15 to 50 amino acids in length.

In some embodiments, about 10⁶, or about 10⁵ or fewer, library members of the anti-phosphoamino acid focused library are screened.

In some embodiments, antibody libraries that are screened in accordance with the methods of the invention are in a Fab, Fab′ or Fv antibody format.

In some embodiments, the anti-phosphoamino acid-focused library employed in the invention is a display library, e.g., a phage display, bacterial display, or yeast display library.

In some embodiments of the invention, the anti-phosphoamino acid-focused library comprises binding members that: bind to the phosphoamino acid to which the reference antibody binds and comprise at least one heavy chain CDR minimal essential binding specificity determinant from the reference anti-phosphoamino acid antibody and at least one light chain CDR minimal essential binding specificity determinant from the reference anti-phosphoamino acid antibody; and have at least one exchange cassette for which members of the library comprise corresponding cassettes that have different sequences.

In another aspect, the invention provides an in vivo method of obtaining an antibody to an epitope of interest, the method comprising: (a) immunizing an animal with a peptide antigen epitope of interest in which a phosphoamino acid has been substituted for a naturally occurring glutamic acid or aspartic acid; and (b) isolating a monoclonal antibody that (i) binds the phosphoamino acid-containing peptide antigen and (ii) binds the epitope of interest that comprises the aspartic acid or glutamic acid.

### BRIEF DESCRIPTION OF THE DRAWINGS

FIG. 1A provides amino acid sequences (SEQ ID NOS:1-5) of peptides employed in Example 1. FIG. 1B provide amino acid sequences (SEQ ID NOS:6-10) of peptides employed in Example 2.

FIG. 2 shows a fractionation protocol used for the polyclonal antibody preparation generated in Example 1.

FIG. 3 shows binding data for the polyclonal antibody 5080-PS fraction to the phosphoserine-labeled KBP0034-BSA conjugate and the serine-containing KBP0035-BSA conjugate.

FIG. 4 shows binding data for the 5080-PS-E polyclonal antibody fraction to the phosphoserine-labeled KBP0034-BSA and the glutamic acid-containing KBP0040-BSA conjugates.

FIGS. 5 A and B provide binding data from antibody preparations from two mice to KBP0049p-BSA conjugate compared to the serine-containing KBP0049-BSA conjugate.

FIGS. 6A to 6E provide binding data for five clones to KBP0049-P, KB0049, KBP0051 and Epha3 protein.

FIG. 7 shows the results of an ELISA assay to determine monoclonal IgG binding to the test peptides. All peptides were biotinylated at the terminal cysteine. All of the biotinylated antigens were bound to a SA-coated plate. Expression media was diluted 1:3 with TBST buffer and added to the plate. After washing away unbound IgG, the bound antibodies were detected with an anti-mouse Fc-AP conjugate and a chemiluminescent substrate for AP.

FIG. 8 shows the sequences of human engineered heavy chain V-regions (SEQ ID NOS:12-21, 18, 22 AND 23, respectively) that support phosphoserine binding. The closest human germ-line V-segment (SEQ ID NO:11) is included for comparison and is underlined.

FIG. 9 shows the sequences of human engineered light chain V-regions (SEQ ID NOS:25-39 and 41-47, respectively) that support phosphoserine binding. The closest human germ-line V-segments for VkI (SEQ ID NO:24) and VkIII (SEQ ID NO:40) are included for comparison and are underlined.

### DETAILED DESCRIPTION OF THE INVENTION

### Definitions

A “phosphoamino acid” as used in the context of this invention refers to a phosphoserine or phosphothreonine, and unnatural amino acid mimetics of phosphoserine and phosphothreonine (see, e.g., U.S. Pat. No. 6,309,863).

A “phosphoamino acid-containing epitope” in the context of this invention refers to a phosphoamino acid, i.e., phosphoserine or phosphothreonine, that is incorporated into an epitope of interest to substitute for a “phosphomimetic” amino acid, e.g., an aspartic acid, or a glutamic acid, that has carbon spacing and charge similar to the phosphoamino acid. The epitope of interest may be a peptide, or the epitope may be present in a longer protein. The phosphomimetic amino acid may be “naturally occurring”, i.e., it is present in a wildtype amino acid sequence of the epitope of interest, or may be introduced into a naturally occurring amino acid sequence.

An “anti-phosphoamino acid-focused library” in the context of this invention refers to a library of antibodies comprising diverse antibody sequences wherein members of the library have a heavy chain that comprises at least one CDR minimal essential binding specificity determinant, e.g., a minimal essential binding specificity determinant from a CDR3, from a reference anti-phosphoamino acid antibody, which reference antibody binds to phosphoserine or phosphothreonine, and/or a light chain that comprises at least one CDR minimal essential binding specificity determinant, e.g., a C minimal essential binding specificity determinant from a CDR3, from the light chain of the reference anti-phosphoamino acid antibody. The members of the library have different sequences relative to one another. When referring to an “antibody library” or “anti-phosphoamino acid-focused library”, the term refers to not only the collection of antibodies produced by the library, but also to the colonies, phage, and the like that express the antibodies. An anti-phosphoamino acid focused library can be from any anti-phosphoserine or anti-phosphothreonine antibody. For example, various mouse monoclonal antibodies directed against phosphoserine and phosphothreonine are commercially available. Antibodies directed against phosphoserine include PSR-45 (Sigma), PS-53 (Novus Biologicals), 106.1 (ThermoPierce), 3C171 (ThermoPierce), 9A354 (US Biological), 6D664 (US Biological) and 11C149 (US Biological). Antibodies directed against phosphothreonine include PTR-8 (Sigma), 5H19 (US Biological), 11C156 (US Biological) and 9A355 (US Biological).

The terms “competitor phosphoamino acid”, “comparator phosphoamino acid”, “phosphoamino acid competitor” and “phosphoamino acid comparator” are used interchangeably herein to refer to the phosphoamino acid in a form where it is not linked to the epitope of interest. Thus, in embodiments, e.g., in which the screening method comprises screening with a comparator phosphoamino acid, this refers to phosphoamino acid that is not linked to the epitope of interest. The phosphoamino acid may, however, be linked to a protein carrier, such as bovine serum albumin, or a non-peptide carrier, e.g., polyethylene glycol (PEG).

A “sub-library” refers to a collection of clones obtained by screening an initial library for a desirable characteristic, e.g., the ability to bind a phosphoamino acid-containing epitope of interest with a higher affinity than the affinity for a comparator phosphoamino acid. In some embodiments, a “sub-library” is subjected to further manipulation prior to screening of the sub-library.

“Repertoire” or “library” refers to a library of genes encoding antibodies or antibody fragments such as Fab, scFv, Fd, V_{H}, or V_{L}, or a subfragment of a variable region, e.g., an exchange cassette, that is obtained from a natural ensemble, or “repertoire”, of antibody genes present, e.g., in human donors, and obtained primarily from the cells of peripheral blood and spleen. In some embodiments, the human donors are “non-immune”, i.e., not presenting with symptoms of infection. In the current invention, a library or repertoire often comprises members that are exchange cassettes of a given portion of a V region. As noted above, the term “library” or “repertoire” refers not only to genes, but to the collection of antibodies or antibody fragments encoded by genes, as well as colonies, phage, and the like that express the antibodies or antibody fragments.

“Synthetic antibody library” refers to a library of genes encoding one or more antibodies or antibody fragments such as Fab, scFv, Fd, V_{H}, or V_{L}, or a subfragment of a variable region, e.g., an exchange cassette, in which one or more of the complementarity-determining regions (CDR) has been partially or fully altered, e.g., by oligonucleotide-directed mutagenesis. “Randomized” means that part or all of the sequence encoding the CDR has been replaced by sequence randomly encoding all twenty amino acids or some subset of the amino acids.

As used herein, an “antibody” refers to a protein functionally defined as a binding protein and structurally defined as comprising an amino acid sequence that is recognized by one of skill as being derived from an immunoglobulin encoding gene of an animal producing antibodies. An antibody can consist of one or more polypeptides substantially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

A typical IgG antibody structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

The term “antibody” as used herein also includes antibody fragments that retain binding specificity and affinity. For example, there are a number of well-characterized antibody fragments. Thus, for example, pepsin digests an antibody C-terminal to the disulfide linkages in the hinge region to produce a F(ab′)₂ fragment, a dimer of Fab which itself is a light chain joined to VH-CH1 by a disulfide bond. The F(ab′)₂ may be reduced under mild conditions to break the disulfide linkage in the hinge region thereby converting the F(ab′)₂ dimer into an Fab′ monomer. The Fab′ monomer is essentially a Fab with part of the hinge region (see, Fundamental Immunology, W. E. Paul, ed., Raven Press, N.Y. (1993), for a more detailed description of other antibody fragments). While various antibody fragments are defined in terms of the digestion of an intact antibody, one of skill in the art will appreciate that fragments are typically synthesized de novo either chemically or by utilizing recombinant DNA methodology. Thus, the term antibody, as used herein, also includes antibody fragments either produced by the modification of whole antibodies or synthesized using recombinant DNA methodologies.

Antibodies include V_{H}-V_{L} dimers, including single chain antibodies (antibodies that exist as a single polypeptide chain), such as single chain Fv antibodies (scFv) in which a variable heavy and a variable light region are joined together (directly or through a peptide linker) to form a continuous polypeptide (e.g., Huston, et al. Proc. Nat. Acad. Sci. USA, 85:5879-5883, 1988). In a single chain antibody format, while the V_{H} and V_{L} are connected to each as a single polypeptide chain, the V_{H} and V_{L} domains associate non-covalently. Alternatively, an antibody can be another fragment. Other fragments can also be generated, e.g., using recombinant techniques, as soluble proteins or as fragments obtained from display methods. Antibodies can also include diabodies (e.g., Holliger, Proc. Natl. Acad. Sci. USA 90:6444-6448, 1993); miniantibodies; heavy chain dimers, such as antibodies from camelids; as well as other antibody formats.

As used herein, “V-region of an antibody” refers to the V_{H} and V_{L}; the V-region of an antibody chain refers to an antibody variable region V_{H} or V_{L} domain comprising the segments of Framework 1, CDR1, Framework 2, CDR2, and Framework 3, CDR3, and Framework 4. The CDR3 and framework 4 are added to the V-segment as a consequence of rearrangement of the heavy chain and light chain V-region genes during B-cell differentiation.

As used herein, “complementarity-determining region (CDR)” refers to the three hypervariable regions in each chain that interrupt the four “framework” regions established by the light and heavy chain variable regions. The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found.

The sequences of the framework regions of different light or heavy chains are relatively conserved within a species. The framework region of an antibody, that is the combined framework regions of the constituent light and heavy chains, serves to position and align the CDRs in three-dimensional space.

The amino acid sequences of the CDRs and framework regions can be determined using various well known definitions in the art, e.g., Kabat, Chothia, international ImMunoGeneTics database (IMGT), and AbM (see, e.g., Johnson et al., supra; Chothia & Lesk, 1987, Canonical structures for the hypervariable regions of immunoglobulins. J. Mol. Biol. 196, 901-917; Chothia C. et al., 1989, Conformations of immunoglobulin hypervariable regions. Nature 342, 877-883; Chothia C. et al., 1992, structural repertoire of the human VH segments J. Mol. Biol. 227, 799-817; Al-Lazikani et al., J. Mol. Biol 1997, 273(4)). Definitions of antigen combining sites are also described in the following: Ruiz et al., IMGT, the international ImMunoGeneTics database. Nucleic Acids Res., 28, 219-221 (2000); and Lefranc, M.-P. IMGT, the international ImMunoGeneTics database. Nucleic Acids Res. January 1; 29(1):207-9 (2001); MacCallum et al, Antibody-antigen interactions: Contact analysis and binding site topography, J. Mol. Biol., 262 (5), 732-745 (1996); and Martin et al, Proc. Natl. Acad. Sci. USA, 86, 9268-9272 (1989); Martin, et al, Methods Enzymol., 203, 121-153, (1991); Pedersen et al, Immunomethods, 1, 126, (1992); and Rees et al, In Sternberg M. J. E. (ed.), Protein Structure Prediction. Oxford University Press, Oxford, 141-172 1996).

“Epitope” as used herein refers to a site on an antigen to which an antibody binds. Epitopes can be formed both from contiguous amino acids or noncontiguous amino acids juxtaposed by tertiary folding of a protein. Epitopes formed from contiguous amino acids are typically retained on exposure of the protein antigen to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed (1996). In the claimed methods, when a library is screened with an epitope of interest, the screening is typically performed with a longer polypeptide, e.g., a protein antigen that includes the epitope of interest. For example, a library may be screened with a peptide, e.g., of 20-25 amino acids, (e.g., when it is desired to obtain an antibody to a linear epitope) that includes the epitope sequence; or may be screened with a large protein antigen that comprises the epitope of interest, e.g., the sequence for which it is desirable to obtain an antibody. The protein antigen may be the protein in which the epitope of interest naturally occurs or may be a heterologous protein, e.g., screening with the epitope of interest may employ the epitope fused to a scaffold polypeptide sequence or other heterologous sequence. The term “screening the library with an epitope of interest” encompasses these various embodiments.

A “complementary variable region” or a “complementary V-region” as used herein refers to a region that can dimerize with a V-region to produce a functional binding fragment that binds selectively to an antigen of interest. A complementary variable region is typically a V_{L} region, where the variable region is a V_{H} region; or is a V_{H} region, where the variable region is a V_{L} region. The complementary variable region often comprises a CDR3 from a reference antibody that binds to the antigen of interest.

The term “V-segment” refers to the region of the V-region (heavy or light chain) that is encoded by a V gene. For example, The V-segment of the heavy chain variable region encodes FR1-CDR1-FR2-CDR2 and FR3. A “D-segment” refers to the region of a V-region that is encoded by a D gene. Similarly, a “J-segment” refers to a region encoded by a J gene. These terms include various modifications, additions, deletions, and somatic mutations that can occur during maturation.

An “exchange cassette” as used herein typically refers to at least one intact CDR adjoined to at least one intact framework region that are together, naturally occurring. An “exchange cassette” also can refer to at least a part of one CDR that is adjoined to at least one framework that are, together, naturally occurring. In other embodiments, an exchange cassette refers to at least one CDR joined to at least a part of one FR that are together, naturally occurring. An “exchange cassette” can also comprise at least one partial CDR adjoined to at least one partial FR that are together, naturally occurring. An “exchange cassette” can also be isolated from a synthetic library in which one or more of the CDRs is mutated. In this case, the CDR prior to mutagenesis and framework region together are naturally occurring. As used herein, a “front” or “front end” cassette contains CDR1 and at least a partial framework region. Accordingly, a “front” cassette has FR1 and CDR1 and may have part or all of FR2. A “middle” cassette as used herein contains CDR2 and at least a partial framework region. Accordingly, a “middle” cassette has CDR2 and FR3 and may have part or all of FR2.

A “partial CDR” or “part of a CDR” or “partial CDR sequence” in the context of this invention refers to a subregion of an intact CDR sequence, e.g., the CDR region outside of the minimal essential binding site, that is present in an exchange cassette. An exchange cassette of this invention can thus have a “partial” CDR. The end result in the hybrid V-region is a hybrid CDR. For example, a CDR2-FR3 exchange cassette includes embodiments in which a subregion of the CDR2 sequence is present in the CDR2-FR3 exchange cassette such that a hybrid V-region resulting from a CDR2-FR3 exchange would have a CDR2 in which part of the CDR2 is from the exchanged cassette and part is from the CDR2 of the reference antibody. A “partial” CDR sequence comprises a subregion of contiguous residues that is at least 20%, typically at least 30%, 40%, 50%, 60%, 70%, 80%, or 90% or more of the intact CDR.

A “minimal essential binding specificity determinant” or “MEBSD” is the region within a CDR sequence, e.g., a CDR3, that is required to retain the binding specificity of the reference antibody when combined with other sequences, typically human sequences, that re-constitute the remainder of a CDR and the rest of the V-region. As appreciated by one of skill in the art, when the reference antibody minimal binding specificity determinant is less than a complete CDR, a complete CDR still results in the anti-phosphoamino acid antibody expression library, as the remaining CDR residues are incorporated into the construct. For example, where the CDR is CDR3, appropriate oligonucleotides can be designed to incorporate human sequences, e.g., germline J segments, to replace the CDR3 residues that are not part of the MEBSD.

A “partial FR” or “part of a FR” or “partial FR sequence” in the context of this invention refers to a subregion of an intact FR that is present in an exchange cassette. Accordingly, an exchange cassette of the invention can have a “partial FR” such that a hybrid V-region that is generated from an exchange cassette that has a partial FR, has part of its FR sequence from the exchanged cassette and part of the FR from the V-region of the reference antibody. A “partial” FR sequence comprises a subregion of contiguous residues that is at least 20%, typically at least 20%, typically at least 30%, 40%, 50%, 60%, 70%, 80%, or 90% or more of the intact FR.

An “extended cassette” as used herein refers to an exchange cassette that comprises an additional framework region. Thus, here an “extended cassette” is an exchange cassette that has at least one CDR and at least two framework regions that are typically, together, naturally occurring. An “extended cassette” can also be isolated from a synthetic library in which one or more of the CDRs is mutated. In this case, the CDR prior to mutagenesis and framework region together are naturally occurring (i.e., typically not altered by recombinant means).

A “corresponding” exchange cassette refers to a CDR and a framework region that is encoded by a different antibody gene or gene segment (relative to an antibody that is to undergo exchange), but is, in terms of general antibody structure, the same CDR and framework region of the antibody. For example, a CDR1-FR1 exchange cassette is replaced by a “corresponding” CDR1-FR1 cassette that is encoded by a different antibody gene relative to the reference CDR1-FR1. The definition also applies to an exchange cassette having a partial CDR sequence and/or a partial FR region sequence.

A “hybrid V region” refers to a V-region in which at least one exchange cassette has been replaced by a corresponding exchange cassette from a different antibody gene or gene segment.

“Antigen” refers to substances that are capable, under appropriate conditions, of inducing a specific immune response and of reacting with the products of the response, that is, with specific antibodies or specifically sensitized T-lymphocytes, or both. Antigens may be soluble substances, such as toxins and foreign proteins, or particulates, such as bacteria and tissue cells; however, only the portion of the protein or polysaccharide molecule known as the antigenic determinant (epitopes) combines with the antibody or a specific receptor on a lymphocyte. More broadly, the term “antigen” may be used to refer to any substance to which an antibody binds, or for which antibodies are desired, regardless of whether the substance is immunogenic. For such antigens, antibodies may be identified by recombinant methods, independently of any immune response.

The “binding specificity” of an antibody refers to the identity of the antigen to which the antibody binds, preferably to the identity of the epitope to which the antibody binds.

The phrase “specifically (or selectively) binds” to an antibody or “specifically (or selectively) immunoreactive with,” when referring to a protein or peptide, refers to a binding reaction where the antibody binds to the protein of interest. In the context of this invention, the antibody typically binds to the phosphamino acid-labeled epitope of interest with an affinity that is at least 2-3-fold better than its affinity for the comparator phosphoamino acid.

The term “equilibrium dissociation constant (K_{D}) refers to the dissociation rate constant (k_{d}, time⁻¹) divided by the association rate constant (kₐ, time⁻¹, M⁻¹). Equilibrium dissociation constants can be measured using any known method in the art. A “high affinity” antibody in the context of this invention has an affinity less than 500 nM, and often less than 50 nM or 10 nM. Thus, in some embodiments, a high affinity antibody has an affinity in the range of 500 nM to 100 pM, or in the range of 50 or 25 nM to 100 pM, or in the range of 50 or 25 nM to 50 pM, or in the range of 50 nM or 25 nM to 1 pM.

The term “increased binding” or “better binding” when comparing binding of an antibody to one molecule, e.g., a phosphoamino acid-containing epitope of interest, vs. another molecule, e.g., a comparator phosphoamino acid or a reference antibody, can result from an increase in binding affinity, an increase in the association rate, or a decrease in the dissociation rate. “Increased binding” or “better binding” is typically reflected by a stronger signal when assessing binding, e.g., via an ELISA.

“Chimeric polynucleotide” means that the polynucleotide comprises regions which are wild-type and regions which are mutated. The term also refers to embodiments in which the polynucleotide comprises wild-type regions from one polynucleotide and wild-type regions from another related polynucleotide.

The term “heterologous” when used with reference to portions of a polynucleotide indicates that the nucleic acid comprises two or more subsequences that are not normally found in the same relationship to each other in nature. For instance, the nucleic acid is typically recombinantly produced, having two or more sequences, e.g., from unrelated genes arranged to make a new functional nucleic acid. Similarly, a “heterologous” polypeptide or protein refers to two or more subsequences that are not found in the same relationship to each other in nature.

“Expression vector” includes vectors which are capable of expressing nucleic acid sequences contained therein, i.e., any nucleic acid sequence which is capable of effecting expression of a specified nucleic acid code disposed therein (the coding sequences are operably linked to other sequences capable of effecting their expression). Some expression vectors are replicable in the host organism either as episomes or as an integral part of the chromosomal DNA. A useful, but not a necessary, element of an effective expression vector is a marker encoding sequence—i.e. a sequence encoding a protein which results in a phenotypic property (e.g. tetracycline resistance) of the cells containing the protein which permits those cells to be readily identified. Expression vectors are frequently in the form of plasmids or viruses. However, the invention is intended to include such other forms of expression vectors which serve equivalent functions and which may, from time to time become known in the art.

“Host cell” refers to a prokaryotic or eukaryotic cell into which the vectors of the invention may be introduced, expressed and/or propagated. A microbial host cell is a cell of a prokaryotic or eukaryotic micro-organism, including bacteria, yeasts, microscopic fungi and microscopic phases in the life-cycle of fungi and slime molds. Typical prokaryotic host cells include various strains of E. coli. Typical eukaryotic host cells are yeast or filamentous fungi, insect cells, or mammalian cells, such as Chinese hamster ovary cells, murine NIH 3T3 fibroblasts, human embryonic kidney 293 cells, or rodent myeloma or hybridoma cells.

“Isolated” refers to a nucleic acid or polypeptide separated not only from other nucleic acids or polypeptides that are present in the natural source of the nucleic acid or polypeptide, but also from polypeptides, and preferably refers to a nucleic acid or polypeptide found in the presence of (if anything) only a solvent, buffer, ion, or other component normally present in a solution of the same. The terms “isolated” and “purified” do not encompass nucleic acids or polypeptides present in their natural source.

“Purified” means that the indicated nucleic acid or polypeptide is present in the substantial absence of other biological macromolecules, e.g., polynucleotides, proteins, and the like. In one embodiment, the polynucleotide or polypeptide is purified such that it constitutes at least 95% by weight, more preferably at least 99.8% by weight, of the indicated biological macromolecules present (but water, buffers, and other small molecules, especially molecules having a molecular weight of less than 1000 daltons, can be present).

“Recombinant” as it relates to a nucleic acid refers to a nucleic acid in a form not normally found in nature. That is, a recombinant nucleic acid is flanked by a nucleotide sequence not naturally flanking the nucleic acid or has a sequence not normally found in nature. Recombinant nucleic acids can be originally formed in vitro by the manipulation of nucleic acid by restriction endonucleases, or alternatively using such techniques as polymerase chain reaction. It is understood that once a recombinant nucleic acid is made and reintroduced into a host cell or organism, it will replicate non-recombinantly, i.e., using the in vivo cellular machinery of the host cell rather than in vitro manipulations; however, such nucleic acids, once produced recombinantly, although subsequently replicated non-recombinantly, are still considered recombinant for the purposes of the invention.

“Recombinant” polypeptide refers to a polypeptide expressed from a recombinant nucleic acid, or a polypeptide that is chemically synthesized in vitro.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers, those containing modified residues, and non-naturally occurring amino acid polymer.

The term “amino acid” refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, γ-carboxyglutamate, and O-phosphoserine.

Preferably, amino acid “substitutions” are the result of replacing one amino acid with another amino acid that typically has similar structural and/or chemical properties, e.g., conservative amino acid replacements. Amino acid substitutions may be made on the basis of similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid.

“Insertions” or “deletions” are typically in the range of about 1 to 5 amino acids. The variation allowed may be experimentally determined by systematically making insertions, deletions, or substitutions of amino acids in a polypeptide molecule using recombinant DNA techniques and assaying the resulting recombinant variants for activity.

Alternatively, where alteration of function is desired, insertions, deletions or non-conservative alterations can be engineered to produce altered polypeptides. Such alterations can, for example, alter one or more of the biological functions or biochemical characteristics of the polypeptides of the invention. For example, such alterations may change polypeptide characteristics such as ligand-binding affinities, interchain affinities, or degradation/turnover rate. Further, such alterations can be selected so as to generate polypeptides that are better suited for expression, scale up and the like in the host cells chosen for expression. For example, cysteine residues can be deleted or substituted with another amino acid residue in order to eliminate disulfide bridges.

Recombinant variants encoding the same polypeptides as an indicated amino acid sequence may be synthesized or selected by making use of the “redundancy” in the genetic code. Various codon substitutions, such as the silent changes which produce various restriction sites, may be introduced to optimize cloning into a plasmid or viral vector or expression in a particular prokaryotic or eukaryotic system. Mutations in the polynucleotide sequence may be reflected in the polypeptide or domains of other peptides added to the polypeptide to modify the properties of any part of the polypeptide, to change characteristics such as ligand-binding affinities, interchain affinities, or degradation/turnover rate.

In the general context of this invention, the term “a” or “an” is intended to mean “one or more”.

### DETAILED DESCRIPTION OF THE INVENTION

### Introduction

The invention provides in vivo and in vitro methods of obtaining monoclonal antibodies that bind to a predetermined epitope of an antigen of interest where the epitope has a phosphomimetic amino acid in the sequence. The method is not constrained by tolerization or self-antigen recognition.

The methods of the invention include screening an anti-phosphoamino acid library where the anti-phosphoamino acid-focused library is derived from a reference antibody to a phosphoamino acid. An anti-phosphoamino acid-focused library can be constructed, for example, in which the antibody members of the library retain a minimal essential binding specificity determinant (MEBSD) from at least one heavy and light chain CDR from the reference antibody other parts of the antibody are diversified. The library can then be screened with the epitope of interest labeled with the phosphoamino acid. Positive clones obtained from the screen that bind to the phosphoamino acid-containing epitope can further be screened with the unlabeled epitope to identify those that having binding activity for the unlabeled epitope. One or more screening cycles can be performed. In some embodiments, the residues in the MEBSD from the reference antibody that are critical to phosphoamino acid binding may be removed.

In some embodiments, an antibody to an epitope of interest is obtained using an anti-phosphoamino acid-focused library where the reference antibody that is used to create the library is an antibody to a phosphoserine.

Any number of anti-phosphoamino acid reference antibodies may be used to construct an anti-phosphamino acid-focused library. Typically, a reference antibody is chosen that binds to a phosphoaminio acid with minimal influence of the surrounding amino acid context. A reference antibody typically has an affinity of better than about 100 nM, e.g., 50-100 nM and a rapid off-rate (kd), for example, an off-rate faster than 10⁻³/s or more preferably at least 5×10⁻³/s. A suitable reference antibody typically has at least two, often at least three, and preferably four, CDRs that can be changed and still retain phosphoamino acid binding specificity.

In some embodiments, an antibody to an epitope of interest is obtained by immunizing an animal (e.g., a mouse or a rabbit) with an antigen containing a phosphoamino acid that has been substituted for a glutamic acid or aspartic acid that occurs in an epitope of interest and isolating antibodies that bind to the phosphoamino acid and bind to a second antigen in which the phosphoamino acid is replaced by glutamic acid or aspartic acid.

### Anti-Phosphoamino Acid Libraries

This section describes construction of anti-phosphoamino acid-focused libraries and screening with a phosphoamino acid-containing epitope of interest.

In order to practice one aspect of the invention, an anti-phosphoamino acid antibody is selected and is used to construct an anti-phosphoamino acid library. Recombinant antibodies derived from the library are then selected, based on (i) ability to bind the phosphoamino acid, and (ii) ability to bind the phosphoamino acid-containing epitope of interest. Those antibodies that exhibit increased binding to the phosphoamino acid-containing epitope of interest compared to binding to the phosphoamino acid alone can then be used to construct libraries for additional rounds of screening until an antibody that has the desired binding properties for the epitope of interest is obtained.

Once an antibody that binds to an epitope of interest is identified in an anti-phosphoamino acid-focused library, the antibody can be subjected to additional rounds of epitope focusing, e.g., additional rounds of cassette exchange, chain replacement, CDR shuffling, CDR mutagenesis and the like to obtain an antibody that retains the binding specificity for the epitope of interest that the selected antibody from the anti-phosphoamino acid-focused library has, but binds to the epitope of interest with an improved affinity (lower dissociation constant) in comparison to the starting antibody selected from the anti-phosphoamino acid-focused library.

An anti-phosphoamino acid-focused library can be obtained using a variety of methods. As understood in the art, the methods described below relating to preparation of an anti-phosphoamino acid-focused library can also be used after the initial screening of the anti-phosphoamino acid-focused library to construct sub-libraries for screening for improved binding characteristics for the epitope of interest.

Further, as additionally explained below, the anti-phosphoamino acid-focused library can be any type of library used to screen antibodies, e.g., a display library such as a phage or bacterial surface display library, or a library where the antibody is secreted.

Anti-phosphoamino acid-focused libraries may be constructed from a reference anti-phosphoamino acid antibody using any method known in the art. In generating the anti-phosphoamino acid-focused library, the members of the library retain at least one minimal essential binding specificity determination (MEBSD) from a CDR from the heavy chain and/or at least one MEBSD from a CDR from the light chain of the reference antibody. In some embodiments, the MEBSD is from the CDR3. In some embodiments, the anti-phosphoamino acid libraries retain at least one MEBSD from a CDR from the heavy chain of the reference antibody and at least one MEBSD from the light chain of the reference antibody. In some embodiments, the MEBSD is from the CDR3s. The anti-phosphoamino acid-focused library may retain additional CDR and/or framework sequences from the reference antibody, e.g., the anti-phosphoamino acid-focused library may comprise a heavy and/or light chain CDR3-FR4 from the reference antibody. In some embodiments, the anti-phosphoamino acid-focused library retains the CDR3s of the reference antibody.

In generating the anti-phosphoamino acid-focused library, portions of the V_{H} and V_{L} sequences of the reference antibody are replaced with sequences from another antibody repertoire to generate an anti-phosphoamino acid-focused library having a diversity of sequences. The sequences introduced into the library are typically from a human repertoire.

The reference antibody may be a non-human antibody, e.g., a murine antibody, or can be from any other species.

As noted above, the MEBSD is the region within a CDR sequence, e.g., a CDR3, that is required to retain the binding specificity of the reference antibody when combined with other sequences, typically human sequences, that re-constitute the remainder of CDR and the rest of the V-region.

The MEBSD can be identified as known in the art (see, e.g., US patent application publication no. 20050255552). In brief, the MEBSD can be defined empirically or can be predicted from structural considerations. For empirical determination, methods such as alanine scanning mutagenesis can be performed on the CDR, e.g., a CDR3, region of a reference antibody (Wells, Proc. Natl. Acad. Sci. USA 93:1-6, 1996) in order to identify residues that play a role in binding to antigen. Additional analyses can include Comprehensive Scanning Mutagenesis, in which each residue of a CDR is replaced, one-at-a-time, with each of the 19 alternative amino acids, rather than just replacement with alanine Binding assays such as colony-lift binding assays, can be used to screen libraries of such mutants to determine those mutants that retain binding specificity. Colonies that secrete antibody fragments with assay signals reduced by at least ten-fold relative to the reference antibody can be sequenced and the DNA sequences used to generate a database of amino acid positions in the CDR that are important for retention of binding. The MEBSD can then be defined as the set of residues that do not tolerate single-site substitution, or which tolerate only conservative amino acid substitution.

An MEBSD can also be determined by deletion analysis in which progressively shorter sequences of a reference antibody CDR are evaluated for the ability to confer binding specificity and affinity. For example, where the CDR is a CDR3, this can be accomplished by substituting the CDR3 residues with progressively longer human sequences, e.g., from a human germline J-segment.

The MEBSD can also be deduced from structural considerations. For example, if the x-ray crystal structure is known, or if a model of the interaction of antibody and antigen is available, the MEBSD may be defined from the amino acids required to form suitable contact with the epitope and to retain the structure of the antigen-binding surface. In some cases, the MEBSD can also be predicted from the primary structure. For example, in V_{H} domains, for instance, the MEBSD of the CDR3 can, in some antibodies, correspond to a D-segment (including any deletions or identifiable N-additions resulting from the re-arrangement and maturation of the reference antibody). Further, software programs such as JOINSOLVER® Souto-Carneiro, et al., J. Immunol. 172:6790-6802, 2004) can be used to analyze CDR3 of immunoglobulin gene to search for D germline sequences.

In some embodiments, an anti-phosphoamino acid-focused library is generated using cassette exchange. The V-gene segment of both the heavy and light chain can be regarded as being comprised of a number of cassettes formed by framework and CDR segments. Thus, the V_{H} and V_{L}-gene segments are each comprised of five “minimal cassettes” (CDR1, CDR2, FR1, FR2, and FR3). The V-regions may additionally be considered to be composed of “exchange cassettes” comprised of two or more minimal cassettes where the exchange cassette includes at least one CDR and at least one FR joined in natural order. Thus, for example, an exchange cassette relating to CDR1 may consist of FR1-CDR1 or FR1-CDR1-FR2. There are nine such exchange cassettes in each V-gene segment, consisting of at least one framework and one CDR (and less than three frameworks) in the appropriate order. The complete V-region includes two additional minimal cassettes, CDR3 and FR4. CDR3-related exchange cassettes include CDR3-FR4 or FR3-CDR-3-FR4.

In some embodiments, the anti-phosphoamino acid-focused library is generated by replacing exchange cassettes of the reference anti-phosphoamino acid antibody with a corresponding exchange cassette, e.g., from a repertoire of human antibody sequences.

The methods comprising replacing an exchange cassette of a variable region of an anti-phosphoamino acid reference antibody with a corresponding exchange cassette from an antibody that is encoded by a different gene can be performed sequentially or concurrently. Thus, in some embodiments, one or more members of an anti-phosphoamino acid-focused library in which one exchange cassette has been replaced by a corresponding library of sequences from other antibody genes can be selected for binding to the phosphoamino acid-labeled epitope (or the epitope of interest; thus providing a sub-library) and the sub-library can be subjected to further rounds of replacing cassettes or otherwise manipulated.

Libraries are typically generated using cloned cassettes of reference antibody sequences and repertoires of human immunoglobulin-derived sequences. The human repertoires can be generated by PCR amplification using primers appropriate for the desired segments from cDNA obtained from peripheral blood or spleen, in which case the repertoires are expected to contain clones with somatic mutations. Alternatively, the repertoires can be obtained by amplification of genomic DNA from non-immune system cells in order to obtain non-mutated, germline-encoded sequences.

An exchange cassette typically has at least one framework and one CDR linked in a natural order and has no more than two frameworks and two CDRs. Examples of exchange cassettes that are often used include:

FR1-CDR1

FR1-CDR1-FR2

FR2-CDR2-FR3

CDR2-FR3, or

FR3-CDR3.

The complete V-region has two additional minimal cassettes (CDR3 and FR4) not present in the V-gene segment. Where desired, these additional cassettes from a reference antibody can also be substituted by sequences from a library of human antibody sequences such that a V-region is generated from entirely human sequences while retaining the antigen binding specificity of the reference antibody.

In some embodiments, a CDR in an exchange cassette is a hybrid CDR. A “hybrid CDR” in the context of this invention refers to a CDR that comprises an MEBSD from a reference antibody and additional sequence in the CDR that is different from the CDR sequence of the reference antibody. The MEBSD sub-sequence can be at any position within the CDR and typically comprises one to several amino acids. A CDR cassette can be constructed using any of the six CDRs contained within V_{H} and V_{L}.

Methods for obtaining diverse antibody libraries suitable for use in the present invention to select high-affinity antibodies are known in the art. For example, in the chain-shuffling technique (Marks, et al., Biotechnology 10:779-83, 1992) one chain of an antibody is combined with a naive human repertoire of the other chain. Chain shuffling can be used to screen diverse sequences for one of the antibody chains while retaining an MEBSD present in the other chain.

Similarly, methods for diversifying one or more CDRs may be used, such as libraries of germline CDRs recombined into a single framework (Soderlind et al., Nature Biotech. 18:852, 2000) or randomized CDRs inserted in consensus frameworks (Knappik et al., J. Mol. Biol 296:57-86, 2000).

In some embodiments, the anti-phosphoamino acid-focused library has a diversity of no larger than about 10⁸ recombinants, e.g., about 10⁷, about 10⁶, about 10⁵, about 10⁴, about 10³ recombinants or fewer. Typically, the number of clones screened is no more than about 10⁵ and is often in the range of about 10³ to about 10⁴ or to about 10⁵.

In other embodiments, e.g., in embodiments in which an anti-phosphoamino acid-focused library is screened with an unlabeled epitope of interest without having previously being screened with the phosphoamino acid-labeled epitope, the library is larger, e.g., the library has a diversity of greater than about 10⁹ recombinants, e.g., about 10¹⁰, about 10¹¹, or about 10¹² recombinants.

In some embodiments, the anti-phosphoamino acid-focused library that is screened in accordance with the invention is constructed using an anti-phosphoserine antibody. Methods for generating antibodies to phosphoserine or phosphothreonine are also well known in the art (See, e.g., U.S. Pat. No. 7,723,069 for methods for incorporating phosphoserine into protein or peptide antigens). Additional methods of synthesizing peptides that incorporate phosphoserine or phosphothreonine are described by Arendt et al., Int. J. Peptide Prot. Res. 33:468-476, 1989; and Arendt and Hargrove, Meth. Mol. Biol. Vol 35, Chapter 9, 187-193, 1994. Such peptides can be used to produce anti-phosphoserine or anti-phosphothreonine antibodies.

Anti-phosphoamino acid antibodies that are typically used do not bind to non-phosphorylated proteins/peptides. Some antibodies may bind broadly to phosphoserine or phosphothreonine residues in different peptide sequences whereas other antibodies bind to phosphoserine or phosphothreonine residues but have some peptide sequence specificity.

Any epitope of interest that contains a phosphomimetic (glutamic acid or an aspartic acid), may be substituted with a phosphoserine or phosphothreonine that replaces the glutamic acid or aspartic acid and used to screen such a library. In typical embodiments, the glutamic acid or aspartic acid that is replaced with a phosphoamino acid is naturally occurring in the epitope amino acid sequence. In some embodiments, an epitope of interest can be modified by incorporation of a phosphoamino acid at a site that is not a naturally occurring glutamic acid or aspartic acid, but that can tolerate substitution of the naturally occurring residue with glutamic acid or aspartic acid. In some embodiments where the epitope is prepared by chemical synthesis, e.g., the synthesis of a small peptide, a phosphoamino acid can be incorporated during synthesis at either the position of a naturally occurring glutamic acid or aspartic acid in the epitope of interest or at another position.

### Antibody Libraries

Antibodies can be expressed using any number of known vectors and expression systems. “Vector” refers to a plasmid or phage or virus or vector, for expressing a polypeptide from a DNA (RNA) sequence. The vector can comprise a transcriptional unit comprising an assembly of (1) a genetic element or elements having a regulatory role in gene expression, for example, promoters or enhancers, (2) a structural or coding sequence which is transcribed into mRNA and translated into protein, and (3) appropriate translation initiation and termination sequences. Structural units intended for use in yeast or eukaryotic expression systems may include a leader sequence enabling extra-cellular secretion of translated protein by a host cell.

Libraries of secreted antibodies or antibody fragments can be expressed in prokaryotic or eukaryotic microbial systems or in the cells of higher eukaryotes such as mammalian cells. The antibody library can be a library where the antibody is an IgG, Fv, a Fab, Fab′, F(ab′)₂, single chain Fv, an IgG with a deletion of one more domains, or any other antibody fragment that includes the V-region.

The antibodies can be displayed on the surface of a virus, cell, spore, virus-like particle, or on a ribosome. For this purpose, one or both chains of the antibody fragment are typically expressed as a fusion protein, for example as a fusion to a phage coat protein for display on the surface of filamentous phage. Alternatively, the antibodies of the antibody library can be secreted from a host cell.

Antibody-expressing host cells or phage are selected by screening with a protein in order to isolate clones expressing antibodies of interest.

In some embodiments, the antibody libraries described herein are expressed as soluble antibodies or antibody fragments and secreted from host cells. For example, the libraries can be expressed by secretion from E. coli or yeast and colonies of cells expressing antigen-binders are revealed by a colony-lift binding assay. Any suitable host cell can be used. Such cells include both prokaryotic and eukaryotic cells, e.g., bacteria, yeast, or mammalian cells.

### Library Screening

### Phosphoamino Acid-Containing Epitope for Screening

An anti-phosphoamino acid-focused library generated using any of the methods described above is screened with the epitope of interest labeled with the phosphoamino acid, where the phosphoamino acid replaces a glutamic acid or aspartic acid in the epitope of interest. Screening may employ the epitope of interest as a peptide, e.g., of 15-25 or more amino acids in length that corresponds to the region of an antigen for which it is desired to obtain an antibody; or as a protein, where the epitope of interest is present in a large protein and the library is screened with the antigen that contains the epitope. In embodiments in which the epitope of interest is used in screening as part of a larger protein, the larger protein need not be the native protein in which the epitope of interest is present. The epitope may be fused to a heterologous amino acid sequence and used for screening.

The phosphoamino acid can be introduced into the epitope using any method known in the art. For example, a phosphoamino acid can be introduced during synthesis using known techniques (e.g., U.S. Pat. No. 7,723,069; Arendt et al., Int. J. Peptide Prot. Res. 33:468-476, 1989; and Arendt and Hargrove, Meth. Mol. Biol. Vol 35, Chapter 9, 187-193, 1994). In some embodiments, a serine or threonine can be introduced into the epitope of interest to substitute for a glutamic acid or aspartic acid and can then be phosphorylated using a serine/threonine specific kinase, to be used in screening. A phosphoserine can be introduced into a protein epitope in vivo, for example see Park, et al. Science 333: 1151-1154 (2011).

In some embodiments, screening with phosphoamino acid-containing epitope is performed in the presence of comparator phosphoamino acid. In such embodiments, the comparator phosphoamino acid is provided linked to a carrier protein such as albumin, keyhole limpet antigen, or a nonprotein carrier.

### Screening

Screening can be performed using an number of known techniques as described above. The following section provides an example of library screening using a microbial expression system.

Filter screening methodologies have been described for detection of secreted antibodies specific for a particular antigen. In one format, the secreted antibody fragments are trapped on a membrane which is probed with soluble antigen (Skerra et al (1991) Anal Biochem. 196:151-5). In this case, bacteria harboring plasmid vectors that direct the secretion of Fab fragments into the bacterial periplasm are grown on a membrane or filter. The secreted fragments are allowed to diffuse to a second “capture” membrane coated with antibody which can bind the antibody fragments (eg anti-immunoglobulin antiserum) and the capture filter is probed with specific antigen. Antibody—enzyme conjugates can be used to detect antigen-binding antibody fragments on the capture membrane as a colored spot. The colonies are re-grown on the first membrane and the clone expressing the desired antibody fragment recovered.

Colony lift binding assays have also been described in which the antibodies are allowed to diffuse directly onto an antigen-coated membrane. Giovannoni et al have described such a protocol for the screening of single-chain antibody libraries (Giovannoni et al., Nucleic Acids Research 2001, Vol. 29, No. 5 e27).

Libraries of secreted antibody fragments can also be screened by ELISA, either using pools of multiple clones or screening of individual clones each secreting a unique antibody sequence. One such method for screening individual clones is described by Watkins et al (1997) Anal. Biochem. 253: 37-45. In this case, microtiter wells were coated with anti-Fab antibody to capture Fab fragments secreted directly in the wells. The Fab samples were then probed with soluble biotinylated antigen followed by detection with streptavidin-alkaline phosphatase conjugates.

Following selection of an antibody from the anti-phosphoamino acid-focused library that binds to the epitope of interest, V-regions from the selected antibody may be subjected to additional rounds of diversification, e.g., by exchange cassette, CDR mutagenesis, chain replacement and the like to improve binding affinity to the epitope of interest. For example, the V-segments, or one or more exchange cassettes within the V-segments of the selected antibody can be replaced with a diversity of the corresponding V-segment or exchange cassette. Further, the selected antibody can be subjected to mutagenesis of one or more CDRs, to identify variants (or the selected antibody) that bind to the epitope of interest.

As explained above, display libraries can also be employed. Such libraries are screened using known techniques. For example, positive clones may be selected using immobilized epitope, e.g., phosphoamino acid-labeled epitope.

The anti-phosphoamino acid-focused libraries employed in the methods of the invention that comprise screening with the antigen without phosphoamino acid labelling without prior screening with phosphoamino acid-containing epitope typically have a diversity of greater than about 10⁶ recombinants

The following examples are provided by way of illustration only and not by way of limitation. Those of skill in the art will readily recognize a variety of non-critical parameters that could be changed or modified to yield essentially similar results.

### EXAMPLES

### Example 1

### Generation of a Rabbit Polyclonal Antibody Population to a Phosphoserine (PS)-Labeled Epitope

This example shows that high affinity polyclonal antibodies directed against the phosphoserine residue of a peptide also bind peptide antigens containing the phosphomimetic amino acid residues glutamic acid and aspartic acid.

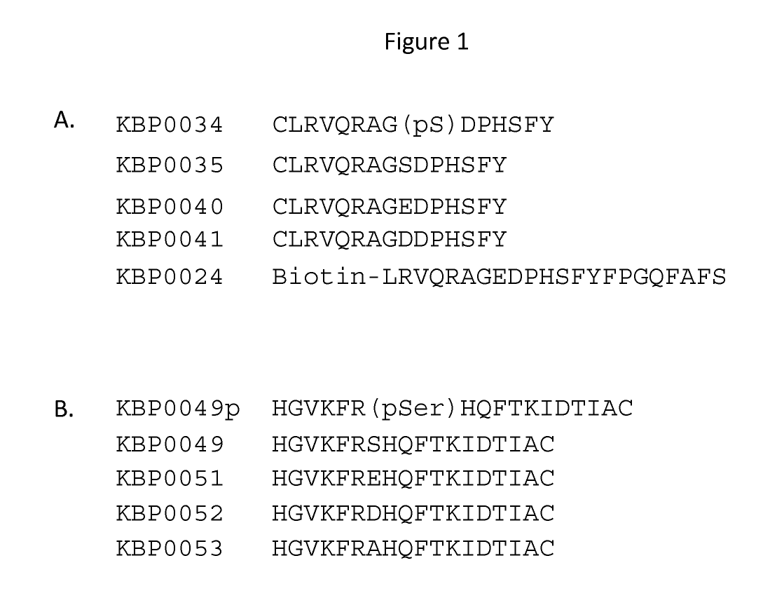

A 15 amino acid region of a protein was synthesized with a phosphoserine residue replacing the wild-type glutamic acid residue at position 143. Additional peptides with serine (KBP0035), glutamic acid (KBP0040), or aspartic acid (KBP0041) were also synthesized. All of the peptides were conjugated to BSA for testing by ELISA analysis. The sequence of each peptide is shown in FIG. 1A.

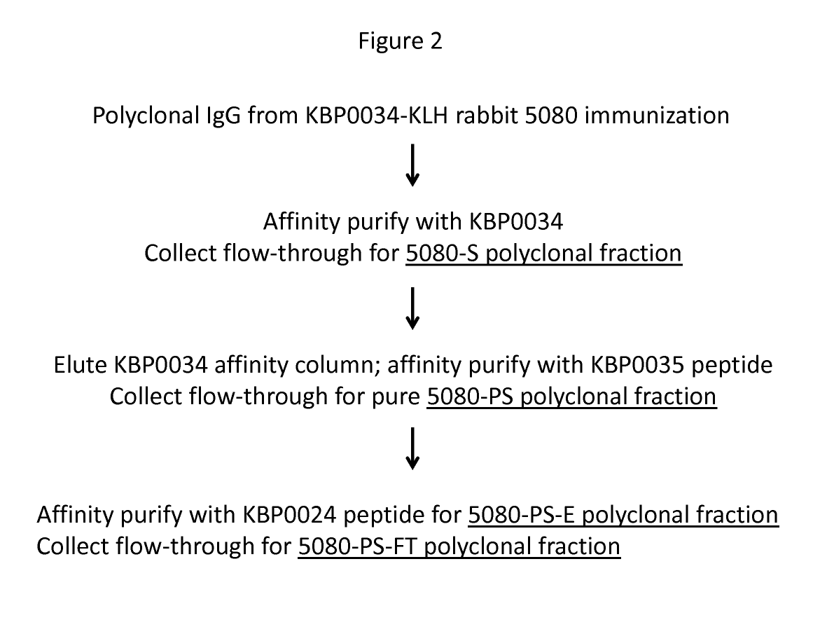

The KBP0034 phosphoserine-labeled peptide was conjugated to a carrier protein keyhole limpet hemocyanin (KLH) and the peptide-KLH conjugate was used to immunize a rabbit. Four immunizations were carried out over a 28-day period. The rabbit was bled and the polyclonal antibody population was fractioned over peptide affinity columns as shown in the flow chart of FIG. 2.

The first two fractions of polyclonal antibody population were used for further testing by ELISA. ELISA plates were coated with the peptide-BSA conjugates and probed with the fractionated rabbit polyclonal antisera. The bound rabbit antibodies were detected with an anti-rabbit secondary antibody labeled with alkaline phosphatase. Phosphoserine-BSA and BSA were included as control antigens. SPM101 is a mouse monoclonal antibody that binds the phosphoserine hapten and was included as a control; the detection of SPM101 was done through an anti-mouse secondary antibody labeled with alkaline phosphatase.

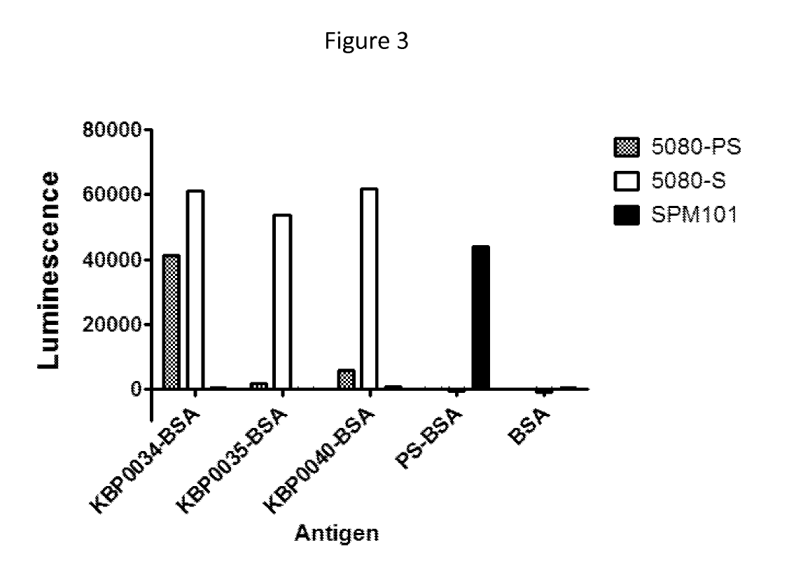

As shown in FIG. 3, polyclonal antibodies from the 5080-PS fraction show strong binding to the phosphoserine-labeled KBP0034-BSA conjugate and minimal binding the serine-containing KBP0035-BSA conjugate; this population is thus comprised of antibodies in which the phosphoserine residue is an essential feature of the epitope. The second population of polyclonal antibodies, 5080-S, shows strong binding to both the KBP0034-BSA and KBP0035-BSA conjugates, indicating it is comprised of antibodies that bind the peptide sequence whether or not phosphoserine is present. Additionally, the 5080-PS polyclonal fraction also shows strong binding to the glutamic acid (wild-type)-containing KBP0040-BSA conjugate indicating that some antibodies in the 5080-PS fraction recognize the phosphomimetic peptide. Antibodies in the 5080-PS fraction did not bind to phosphoserine-BSA indicating that the antibodies that bind to KBP0034-BSA contact the phosphoserine residue as well as the amino acid residues that surround phosphoserine. Neither the 5080-PS or 5080-S antibody pools bound the negative control antigen BSA.

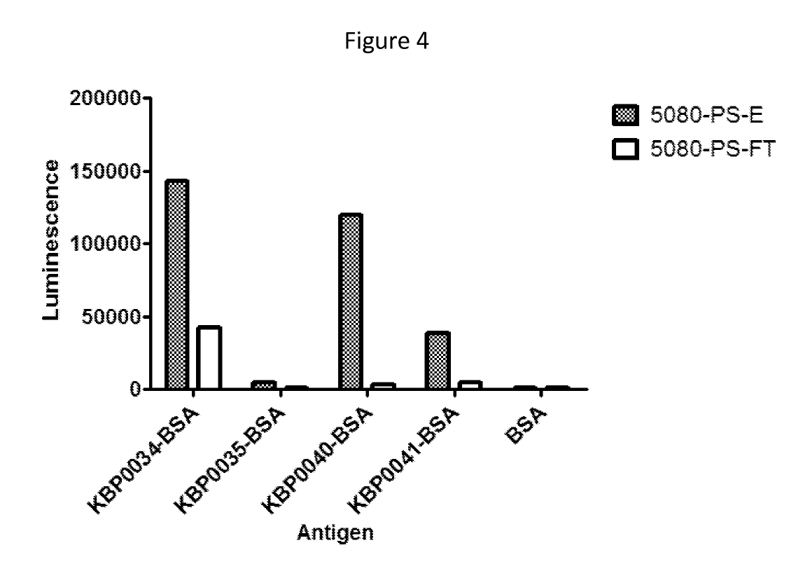

In order to enrich for antibodies that bound the wild-type, glutamic acid peptide antigen, the 5080-PS fraction was applied to an affinity column composed of the biotinylated KBP0024 peptide bound to streptavidin agarose (FIGS. 1A and 2); KBP0024 is a 22-mer peptide that contains the 15-mer KBP0040 sequence and seven additional C-terminal amino acids from the wild-type chemerin sequence. The KBP0024-binding fraction was named 5080-PS-E and the flow-through fraction was named 5080-PS-FT. Both fractions were tested for antigen binding in an ELISA assay. As shown in FIG. 4, the 5080-PS-E polyclonal fraction shows strong binding to both the phosphoserine-labeled KBP0034-BSA and the glutamic acid-containing KBP0040-BSA conjugates. That the binding activities co-enrich indicates that there is a subset of antibodies that can bind peptide antigen containing either phosphoserine or its phosphomimetic glutamic acid. The 5080-PS-E fraction also shows strong binding to the aspartic acid-containing KBP0041-BSA conjugate, indicating that aspartic acid also is a phosphomimetic amino acid to phosphoserine.

Taken together, the results suggest that the 5080-PS rabbit polyclonal antibody fraction contains high affinity antibodies directed against the phosphoserine residue of KBP0034 that also bind peptide antigens containing the phosphomimetic amino acid residues glutamic acid and aspartic acid.

### Example 2

### Generation of Mouse Monoclonal Antibodies to a Phosphoserine (PS)-Labeled Epitope

This example shows that monoclonal antibodies to a phosphoserine-labeled eptiope also bind an epitope in which phosphoserine is replaced by glutamic acid.

For a second illustrative antigen, a 17 amino acid region from the extracellular domain (ECD) of the EphA3 receptor protein (GenBank accession number NP005224.2; positions 131-147) was chosen as an antigen. The 17 amino acid sequence is identical in both the human and mouse EphA3 protein sequences. A peptide, KBP0049p, was synthesized with a phosphoserine residue replacing the wild-type glutamic acid at position 137. Additional peptides with serine (KBP0049) or glutamic acid (KBP0051) at position 137 were also synthesized. All of the peptides contained an additional cysteine residue at the C-terminus for conjugation to BSA. The sequence of each peptide is shown in FIG. 1B. KBP0049p was conjugated to the carrier protein KLH for immunization and all of the peptides were conjugated to BSA for ELISA analysis.

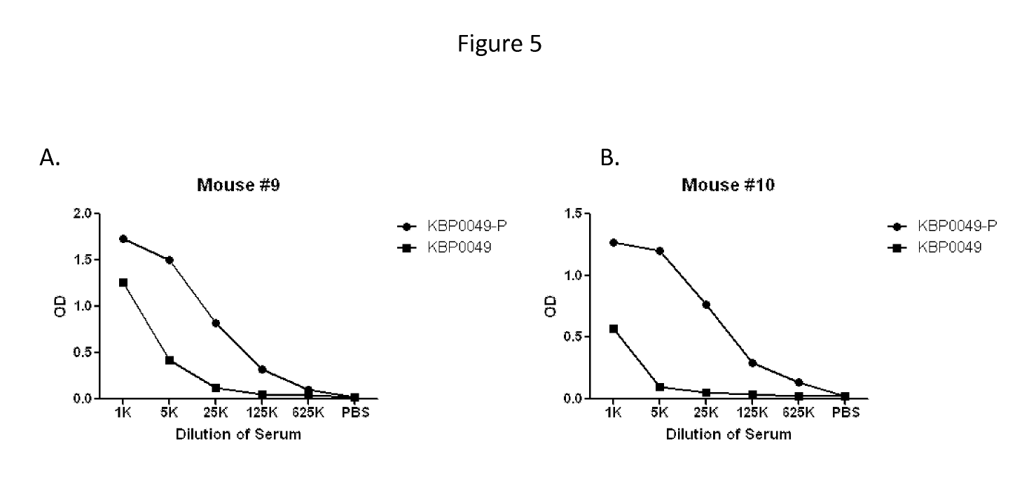

The KBP0049p phosphoserine-labeled peptide-KLH conjugate was used to immunize several mice. Four immunizations were carried out over a 60-day period. The mice were bled and serum containing the polyclonal antibody population was tested by ELISA for binding to the KBP0049p-BSA and KBP0049-BSA conjugates. The ELISA results shown in FIGS. 5A and 5B indicate that mouse #9 and mouse #10 had a higher titer to the KBP0049p-BSA conjugate compared to the serine-containing KBP0049-BSA conjugate.

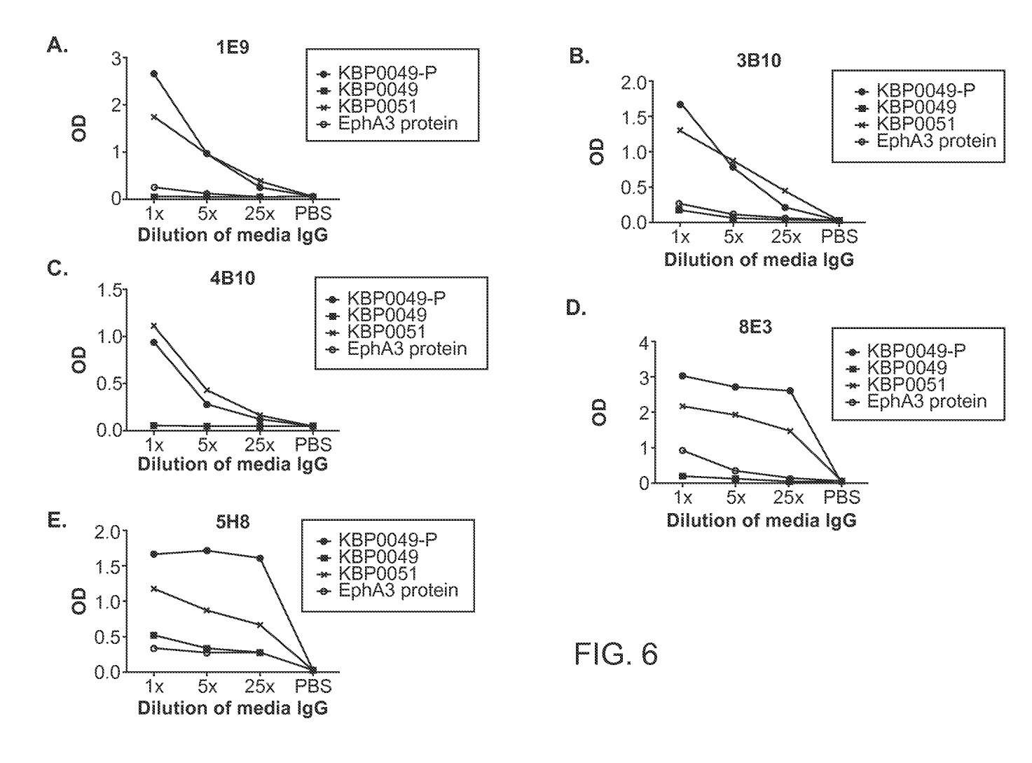

Mouse #9 was given one additional immunization with the KBP0049p-KLH conjugate and mouse #10 was given one additional immunization with the EphA3 receptor ECD protein (521 amino acids). After one week, the spleens were removed and fused with the myeloma line SP2/0. The fused cell lines were expanded and expression media containing IgG was tested in an ELISA for binding to the KBP0049p-BSA and KBP0049-BSA conjugates. As shown in FIG. 6A to 6E, five clones were identified that show strong binding to the KBP0049p-BSA conjugate and low or no detectable binding to the KBP0049-BSA conjugate. Clones 1E9, 3B10, 4B10 and 8E3 were selected from the fusion with mouse #9 and clone 5H8 was selected from the fusion with mouse #10. For each of the monoclonal antibodies, the strong binding to the phosphoserine-containing KBP0049p-BSA conjugate but not to the serine-containing KBP0049-BSA conjugate suggests that phosphoserine is an essential feature of the antibody binding epitope. None of the monoclonal antibodies show binding to the BSA carrier protein (not shown).

The five monoclonal antibodies were also tested for binding to the glutamic acid-containing KBP0051-BSA conjugate and the EphA3 receptor ECD protein. All of the monoclonal antibodies bind strongly to the phosphomimetic, glutamic acid-containing KBP0051-BSA conjugate, some with near identical affinity. Additionally, the six monoclonal antibodies all show binding to the EphA3 protein that contains the 17 amino acid sequence of KBP0051.

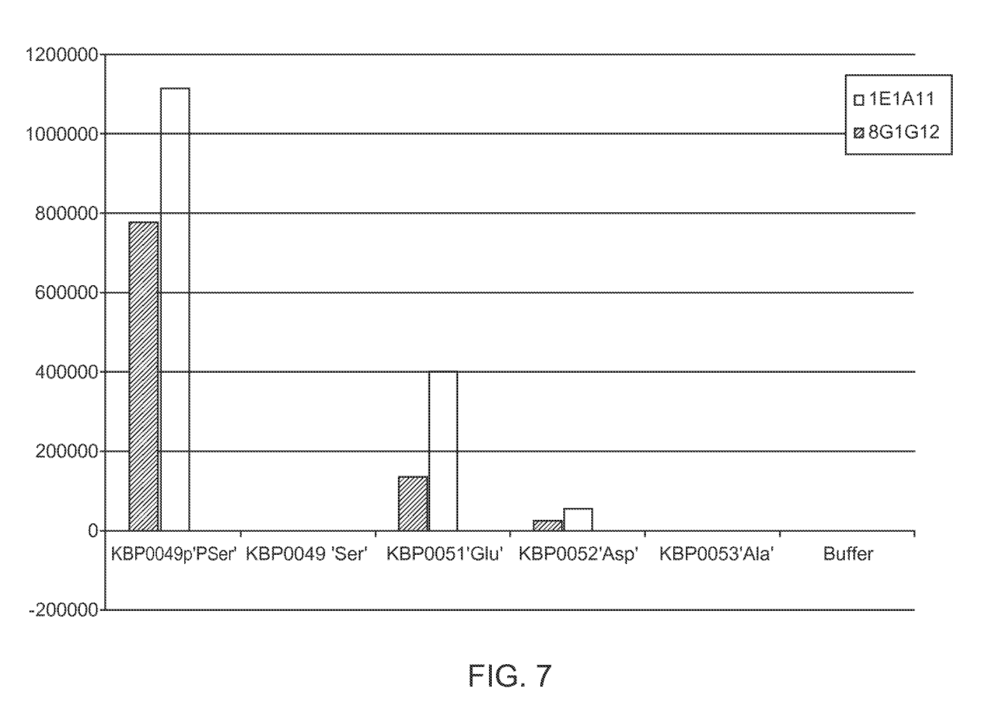

Two of the monoclonal antibodies were sub-cloned and expression media from each was tested by ELISA and ForteBio Octet interferometry. 1E1A11 was sub-cloned from the 1E9 parent cell line and 8G1G12 was sub-cloned from 8E3. Expression media containing IgG from each clone was tested for antigen binding. The KBP0049p, KBP0049, KBP0051, KBP0052 and KBP0053 peptides were conjugated to a maleimide-PEG2-biotin linker (ThermoPierce). An ELISA plate was coated with streptavidin and the biotinylated peptides were bound by the streptavidin. 0.05 ml of expression media from each clone was added to the plate and incubated. After washing away the unbound IgG, the bound IgG was detected with an anti-murine Fc-AP conjugate. A chemiluminescent substrate for AP was added and the emitted light was detected with a plate reader. The results of the ELISA are shown in FIG. 7. The 1E1A11 and 8G1G12 IgGs show strong binding to KBP0049p and weak or undetectable binding to KBP0049, indicating that phosphoserine is a critical feature of the epitope for each IgG. Each of the IgGs also shows binding to the phosphomimetic amino acid aspartic acid and only weak binding to the peptide containing alanine

ForteBio Octet biolayer interferometry was used to assess the binding kinetics of the 1E1A11 and 8G1G12 IgGs. Streptavidin sensors were coated with biotinylated peptide antigen. Each expression media was diluted 10-fold in 1× Kinetics buffer (ForteBio) and used as the analyte. The off-rate kinetic value for each IgG tested on several peptide antigens is shown in Table 1. The kinetic assay and ELISA results are consistent and both show that each IgG strongly binds the KBP0049p (PSer), KBP0051 (Glu) and KBP0052 (Asp) peptides and bind undetectably or weakly to the KBP0049 (Ser) and KBP0053 (Ala) peptides.

TABLE 1
ForteBio Octet kinetic analysis of monoclonal IgG off-rates.
Biotinylated peptides were bound to streptavidin sensors and
media containing monoclonal IgG was flowed over the sensor. The
bivalent off-rate values for each monoclonal IgG are shown.

| | 1E1A11 | 8G1G12 |
|---|---|---|
| Peptide | k_{d} (1/sec) | k_{d} (1/sec) |
| KBP0049p (PSer) | 3.40E−3 | 2.56E−3 |
| KBP0049 (Ser) | NM | NM |
| KBP0051 (Glu) | 4.41E−3 | 1.88E−2 |
| KBP0052 (Asp) | 5.09E−3 | 2.76E−2 |
| KBP0053 (Ala) | NM | NM |

NM = not measurable

Taken together the data show that the phosphoserine-labeled KBP0049p peptide provoked an immune response in the mouse, breaking immunological self-tolerance. The five monoclonal antibodies tested show strong binding to the phosphoserine-labeled KBP0049p-BSA and phosphomimetic KBP0051-BSA conjugates and minimal or no binding to the serine-containing KBP0049-BSA conjugate. The five monoclonal antibodies also show binding to the EphA3 receptor ECD protein. The mouse monoclonal antibodies targeted to a phosphoserine-labeled epitope also bind an epitope in which phosphoserine is replaced by glutamic acid.

### Example 3

### Characterization of V-Region Amino Acid Residues Necessary for Phosphoserine (PS) Binding in Antibody PSR-45

Several mouse monoclonal antibodies directed against phosphoserine and phosphothreonine have been described (PSR-45 (Sigma), PS-53 (Novus Biologicals), 106.1 (ThermoPierce), 3C171 (ThermoPierce), 9A354 (US Biological), 6D664 (US Biological) and 11C149 (US Biological)). Antibodies directed against phosphothreonine include PTR-8 (Sigma), 5H19 (US Biological), 11C156 (US Biological) and 9A355 (US Biological). In addition to binding the isolated phosphoamino acid, the anti-phosphoserine and anti-phosphothreonine antibodies also bind phosphoserine and phosphothreonine, respectively, when the phosphoamino acid is incorporated into a peptide or a protein.

Antibody PSR-45 (Sigma) is a mouse monoclonal antibody which binds to phosphoserine. PSR-45 will also bind PSer when the amino acid is incorporated into peptides or proteins having diverse sequences (e.g. Kim, S.-J. et al. J. Biol. Chem. 279:50031 [2004]; Naz, R. K. Biol. Reproduction 60:1402 [1999]; Gertsberg, I. et al. J. Gen. Physiol. 124:527 [2004]). Thus, PSR-45 binds to PSer independent of the surrounding amino acid context. The PSR-45 V-regions were PCR-amplified from cDNA made from ascites fluid and sequenced.

The CDR regions of PSR-45 are indicated in bold/underline in the following schematics.

PSR-45 light chain V-region
(SEQ ID NO: 48)
EIVLTQSPAIMSASPGEKVTLTCSASSSISDIYWYQQKPGTSPKRWIYD
TSKLSSGVPTRFSGSGSGTSYSLTISSLEAEDAATYYCHQRSNYPYTFG
GGAKLEIK
PSR-45 heavy chain V-region
(SEQ ID NO: 49)
EVQLVESGGGLVQPKGSLKLSCAASGFSIYTYALFWVRQAPGKGLEWVA
RIRSRSKNYATYYADSVKDRFTVSRVDSRNMVYLQMTHLKTEDSAIYYC
VLWSYSRALDYWGQGTSVTVSS

Diverse, human PSer binding libraries were prepared in which important amino acids are either present in the human CDRs or are engineered into all CDRs of the library. Thus, CDRs can be changed in order to retain PSer binding, but add new antibody-antigen contacts in order to add specificity and affinity. V-region constructions and screening for new antigen contacts was performed as follows.

The light and heavy chain V-regions from PSR-45 were cloned into Fab expression vectors containing human constant regions and a 6×His (H6) tag at the end of the CH1 constant region; PSR-45 has a kappa light chain. The FR4 sequence of the heavy and light chains were altered to be identical to human JH6 and human Jk2, respectively, and the optimized reference (opRef) Fab was named MI17-6. For Fab expression in E. coli, the heavy and light chain translation units are not preceded by a signal peptide, but are secreted into the periplasm by the co-expression of a SecY^{mut} gene; the signal-less secretion system has been described in US patent application publication no. 20070020685. The Fab expression plasmid MI17-6 was transformed into the E. coli strain TOP10 along with plasmid KB5282, which expresses the SecY^{mut} gene. The MI17-6 Fab was expressed and secreted into the periplasm and passes into the growth media by passive diffusion.

In order to determine the CDR3 minimal essential binding specificity determinant regions, the HCDR3 and LCDR3 were mutated. Degenerate codons (NNK) were introduced into the HCDR3 and the LCDR3 to construct libraries where each variant differed from the reference CDR3 at only one position. The heavy and light chain CDR3 libraries were appended to the reference and optimized reference V-segments and a library was constructed in which the LCDR3 and HCDR3 variants were randomly mixed. The resulting CDR3 library was expressed in E. coli and screened with an ELISA assay for Fabs binding to phosphoserine-conjugated BSA (PSer-BSA). Many positive clones were detected and several were chosen for sequencing. Table 2 shows the CDR3 sequences for the highest affinity clones.

TABLE 2
HCDR3 and LCDR3 sequences that support
phosphoserine binding (SEQ ID NOS: 51-67).

| | |
|---|---|
| PSR-45 HCDR3 | WSYSRALDY |
| MI20-F8-VH | WSHSRARDY |
| MI20-C17-VH | WSYSRFLDY |
| MI20-H22-VH | WSHSRAMDY |
| MI20-H23-VH | WSHSRALDY |
| MI20-B30-VH | WSYSRSLDY |
| MI20-C32-VH | WSYSRNLDY |
| M120-G38-VH | WSQSRALDY |
| M120-D45-VH | WSYSRGLHY |
| M120-B48-VH | WSYSRAADY |
| PSR-45LCDR3 | HQRSNYPYT |
| MI21-B7-VK | HQRSVYPYT |
| MI21-B10-VK | HQRVNYPYT |
| MI21-D7-VK | HQRSGYPYT |
| MI21-F6-VK | HQRSNVPYT |
| MI21-F10-VK | HQRSFYPYT |
| MI21-G7-VK | HQRTNYPYT |

The mutated amino acid(s) are underlined; in some instances an additional mutation to the CDR3 sequence occurred during PCR amplification.

Humaneering libraries have been described in US patent application publication no. 20050255552. Briefly, the CDR3 BSD region from a reference antibody (typically along with a human germ-line FR4) is appended to a diverse human V-segment library. The resulting library is focused on the same epitope as the reference antibody.

Hybrid cassette libraries were also constructed. Cassette construction has been described in US patent application publication no. 20060134098. Briefly, a cassette is a CDR region along with one or more full or partial flanking human FR regions. For example, a ‘front’ cassette contains a human FR1, CDR1 and a full or partial FR2. A ‘middle’ cassette contains a full or partial human FR2, CDR2 and FR3. A CDR3/FR4 cassette contains the CDR3 and a full or partial FR4. In each case, the ‘front’ or ‘middle’ cassette is joined with the complementary cassette and the CDR3 BSD/FR4 cassette. Cassettes can be a single sequence or a diverse library of sequences. Cassettes are fused together using overlap extension PCR or ligation. The resulting cassette libraries were cloned into Fab expression vectors with the complementary reference or optimized reference chains.

In addition to cassette libraries, HCDR1, HCDR2, LCDR1 and LCDR2 diversity libraries were constructed by replacing some reference amino acids with one or more cognate amino acids from human germ-line sequence. The heavy chain library is based on the Vh3 germ-line subclass and the light chain libraries are based on the VkI or VkIII germ-line subclasses. The diversity libraries were made by synthesizing oligomers with degenerate nucleic acid sequence so that all amino acid combinations are represented in the library. The CDR libraries were joined with selected human FR3-CDR3-FR4 sequences, germ-line FR2 sequences and libraries of FR1 sequences from spleen by overlap extension PCR. The cassette libraries were combined into full V-regions by overlap extension PCR and contained the reference CDR3-human FR4 cassette.

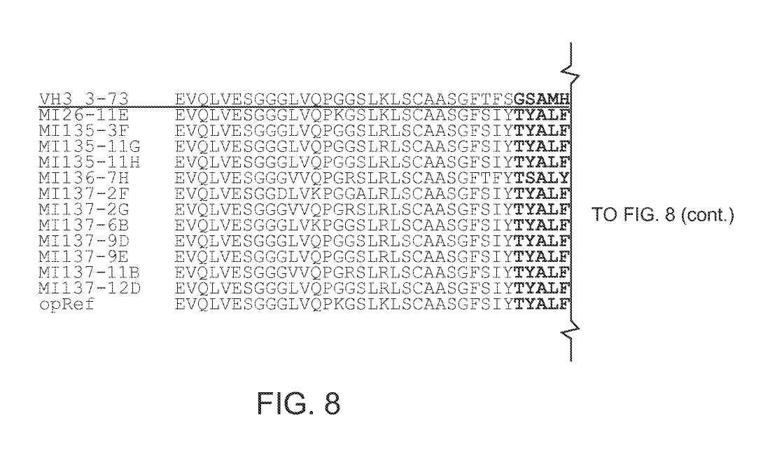

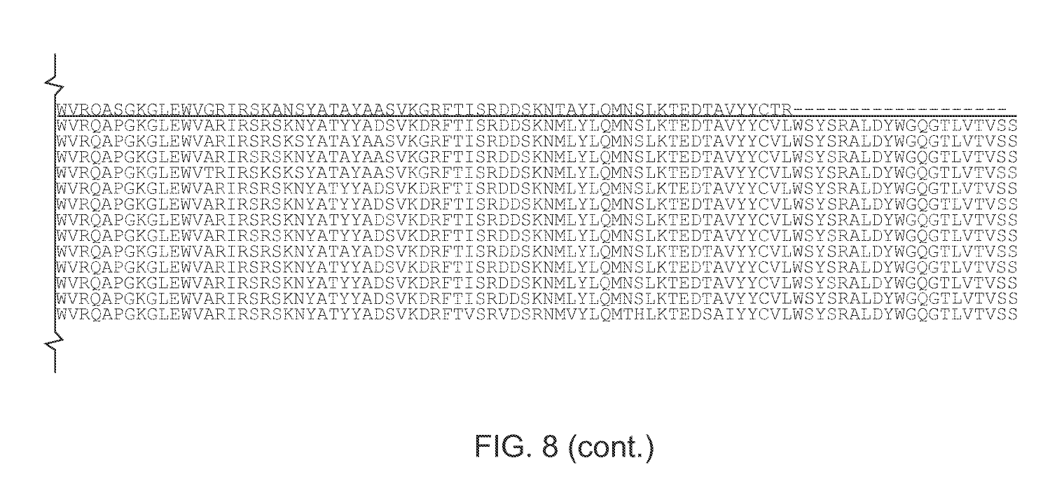

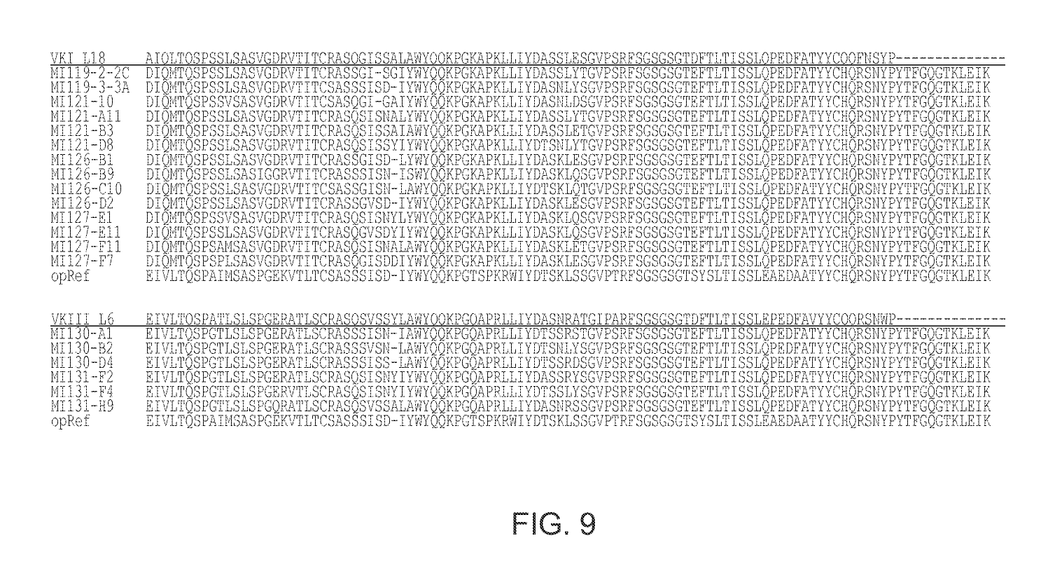

The cassette Fab libraries were screened for binding to PSer-BSA by the plate ELISA assay. Many Fab clones were identified that bound PSer-BSA and the heavy and light chains were sequenced. The sequencing results for the heavy and light chains are shown in FIGS. 8 and 9, respectively. Several heavy chain ‘front’ and ‘middle’ cassettes were identified, indicating that diverse human HCDR1, HCDR2 and adjacent FR sequences can support PSer-BSA binding. Similarly, several ‘front’ and ‘middle’ cassettes were identified for both the VkI and VkIII germ-line subclasses indicating that diverse human LCDR1, LCDR2 and adjacent FR sequences can support PSer-BSA binding.

The results from the heavy and light chain cassette and V-region screens indicate that many diverse human V-segment and CDR3/FR4 cassette sequences support PSer-BSA binding. In order to create a highly diverse library of Humaneered Fabs that bind PSer-BSA, the heavy and light chain cassettes that support PSer-BSA binding were combined in all combinations. The resulting library, MI150, contained >10⁶ Fab sequence combinations, >3% of which bind PSer-BSA with high affinity.

The epitope from an antigen of interest will typically contain a glutamic acid or an aspartic acid residue. The glutamic acid or aspartic acid residue is replaced by a phosphoamino acid (i.e. phosphoserine or phosphothreonine) by any number of methods known to one skilled in the art, including peptide synthesis, in vitro labeling with a protein kinase or in vivo incorporation by protein synthesis. The diverse, phosphoamino acid focused library is contacted with the phosphoamino acid-containing antigen and antibodies that bind the antigen are selected. Those antibodies that exhibit increased binding to the phosphoamino acid-containing epitope of interest compared to binding to the phosphoamino acid alone can then be used to construct libraries for additional rounds of screening until an antibody that has the desired binding properties for the epitope containing the non-phosphorylated residue (i.e. the phosphomimetic glutamic acid or aspartic acid) is obtained.

All patents, patent applications, and other published reference materials cited in this specification are hereby incorporated herein by reference in their entirety for their disclosures of the subject matter in whose connection they are cited herein.

What is claimed is:

1. A method of obtaining an antibody to an epitope of interest containing an aspartic acid or glutamic acid, the method comprising:
(a) obtaining an anti-phosphoamino acid-focused antibody library generated from a reference antibody, wherein the members of the library retain at least one minimal essential binding specificity determinant of a CDR from the reference antibody V_{H} or V_{L};
(b) screening the anti-phosphoamino acid-focused antibody library with a phosphoamino acid-containing epitope, wherein the epitope of interest has a phosphoamino acid substituted for the glutamic acid or for the aspartic acid;
(c) identifying members of the anti-phosphoamino acid-focused antibody library that exhibit better binding to the phosphoamino acid-containing epitope in comparison to the reference antibody, which defines a sublibrary of the anti-phosphoamino acid-focused library;
(d) screening the sublibrary of step (c) with the epitope of interest that has the glutamic acid or aspartic acid; and
(e) selecting an antibody from screening step (d) that binds to the epitope of interest.

2. The method of claim 1, wherein step (b) further comprises screening the anti-phosphoamino acid-focused antibody library with a phosphoamino acid comparator to identify members that exhibit better binding to the phosphamino acid-containing epitope than to the phosphamino acid comparator.

3. The method of claim 1, wherein the members of the anti-phosphoamino acid-focused library retain a minimal essential binding specificity determinant from the reference antibody CDR3 V_{H} or V_{L} region.

4. The method of claim 1, wherein the members of the anti-phosphoamino acid-focused library retain a minimal essential binding specificity determinant from the reference antibody heavy chain CDR3 and a minimal essential binding specificity determinant from the reference antibody light chain CDR3.

5. The method of claim 4, wherein the members of the anti-phosphoamino acid-focused library retain the reference antibody heavy chain CDR3 and the reference antibody light chain CDR3.

6. The method of claim 1, wherein the phosphoamino acid is phosphoserine or phosphothreonine.

7. The method of claim 1, wherein the aspartic acid or glutamic acid is a naturally occurring amino acid in the amino acid sequence of the epitope of interest.

8. A method of obtaining an antibody to an epitope of interest containing an aspartic acid or glutamic acid, the method comprising:
(a) obtaining an anti-phosphoamino acid-focused antibody library generated from a reference antibody, wherein the members of the library retain at least one minimal essential binding specificity determinant of a CDR from the reference antibody V_{H} or V_{L};
(b) screening the anti-phosphoamino acid-focused antibody library with a phosphoamino acid-containing epitope, wherein the epitope of interest has a phosphoamino acid substituted for the glutamic acid or for the aspartic acid;
(c) identifying members of the anti-phosphoamino acid-focused antibody library that exhibit better binding to the phosphoamino acid-containing epitope in comparison to the reference antibody, which defines a sublibrary of the anti-phosphoamino acid-focused library;
(d) selecting one of the V regions of an antibody chain of an antibody identified in step (c) and exchanging a cassette of the selected V region with a library of corresponding cassettes to provide a library of engineered V regions, wherein the selected V region retains at least one minimal essential binding specificity determinant of a CDR from the antibody identified in step (c);
(e) pairing the V region library of step (d) with a complementary V region, or a diverse library of complementary V regions, wherein the complementary V region or the diverse library of complementary V regions comprise an MEBSD from the reference antibody;
(f) screening the library of step (e) with the epitope of interest that has the glutamic acid or aspartic acid; and
(g) selecting an antibody that binds to the epitope wherein the antibody comprises an engineered V region.

9. The method of claim 8, wherein step (b) further comprises screening the anti-phosphoamino acid-focused antibody library with a phosphoamino acid comparator to identify members that exhibit better binding to the phosphamino acid-containing epitope than to the phosphamino acid comparator.

10. The method of claim 8, wherein the diverse library of complementary V regions in step (e) comprises members that have at least one exchange cassette exchanged with corresponding cassettes that have diverse sequences.

11. The method of claim 8, wherein the selected V region of step (d) is a heavy chain V region.

12. The method of claim 8, wherein the cassette that is exchanged in step (d) is a CDR3-FR4 cassette.

13. The method of claim 8, wherein the members of the anti-phosphoamino acid-focused library retain a minimal essential binding specificity determinant from the reference antibody CDR3 V_{H} or V_{L} region.

14. The method of claim 8, wherein the members of the anti-phosphoamino acid-focused library retain a minimal essential binding specificity determinant from the reference antibody heavy chain CDR3 and a minimal essential binding specificity determinant from the reference antibody light chain CDR3.

15. The method of claim 14, wherein the members of the anti-phosphoamino acid-focused library retain the reference antibody heavy chain CDR3 and the reference antibody light chain CDR3.

16. The method of claim 8, wherein the phosphoamino acid is phosphoserine or phosphothreonine.

17. The method of claim 8, wherein the aspartic acid or glutamic acid is a naturally occurring amino acid in the amino acid sequence of the epitope of interest.

18. A method of obtaining an antibody to an epitope of interest containing an aspartic acid or glutamic acid, the method comprising:
(a) obtaining an anti-phosphoamino acid-focused antibody library generated from a reference antibody, wherein the members of the library retain at least one minimal essential binding specificity determinant of a CDR from the reference antibody V_{H} or V_{L};
(b) screening the anti-phosphoamino acid-focused antibody library with a phosphoamino acid-containing epitope, wherein the epitope of interest has a phosphoamino acid substituted for the glutamic acid or for the aspartic acid;
(c) identifying members of the anti-phosphoamino acid-focused antibody library that exhibit better binding to the phosphoamino acid-containing epitope in comparison to the reference antibody, which defines a sublibrary of the anti-phosphoamino acid-focused library;
(d) selecting one of the V regions of an antibody chain of an antibody identified in step (c) and pairing the V region with a diverse library of complementary V regions to form a library of antibodies;
(e) screening the library of step (d) with the epitope of interest that has the glutamic acid or aspartic acid; and
(f) selecting an antibody that binds to the epitope of interest.

19. The method of claim 18, wherein step (b) further comprises screening the anti-phosphoamino acid-focused antibody library with a phosphoamino acid comparator to identify members that exhibit better binding to the phosphamino acid-containing epitope than to the phosphamino acid comparator.

20. The method of claim 18, wherein the V region selected in step (d) is a V_{H} region.

21. The method of claim 18, wherein the members of the anti-phosphoamino acid-focused library retain a minimal essential binding specificity determinant from the reference antibody CDR3 V_{H} or V_{L} region.

22. The method of claim 18, wherein the members of the anti-phosphoamino acid-focused library retain a minimal essential binding specificity determinant from the reference antibody heavy chain CDR3 and a minimal essential binding specificity determinant from the reference antibody light chain CDR3.

23. The method of claim 22, wherein the members of the anti-phosphoamino acid-focused library retain the reference antibody heavy chain CDR3 and the reference antibody light chain CDR3.

24. The method of claim 18, wherein the phosphoamino acid is phosphoserine or phosphothreonine.

25. The method of claim 18, wherein the aspartic acid or glutamic acid is a naturally occurring amino acid in the amino acid sequence of the epitope of interest.

26. The method of claim 1, wherein the anti-phosphoamino acid focused library comprises binding members that:
bind to the phosphoamino acid to which the reference antibody binds and comprise at least one heavy chain CDR minimal essential binding specificity determinant from the reference anti-phosphoamino acid antibody and at least one light chain CDR minimal essential binding specificity determinant from the reference anti-phosphoamino acid antibody; and have at least one exchange cassette for which members of the library comprise corresponding cassettes that have different sequences.

27. A method of obtaining an antibody to an epitope of interest, the method comprising:
(a) immunizing an animal with a peptide antigen epitope of interest in which a phosphoamino acid has been substituted for a naturally occurring glutamic acid or aspartic acid;
(b) isolating a monoclonal antibody that (i) binds the phosphoamino acid-containing peptide antigen and (ii) binds the epitope of interest that comprises the aspartic acid or glutamic acid.


SEQUENCE LISTING (67 sequences)

SEQ ID NO: 1 - protein, 15 aa, Artificial Sequence
  synthetic KBP0034 peptide
CLRVQRAGSDPHSFY

SEQ ID NO: 2 - protein, 15 aa, Artificial Sequence
  synthetic KBP0035 peptide
CLRVQRAGSDPHSFY

SEQ ID NO: 3 - protein, 15 aa, Artificial Sequence
  synthetic KBP0040 peptide
CLRVQRAGEDPHSFY

SEQ ID NO: 4 - protein, 15 aa, Artificial Sequence
  synthetic KBP0041 peptide
CLRVQRAGDDPHSFY

SEQ ID NO: 5 - protein, 22 aa, Artificial Sequence
  synthetic KBP0024 peptide
LRVQRAGEDPHSFYFPGQFAFS

SEQ ID NO: 6 - protein, 18 aa, Artificial Sequence
  synthetic EphA3 receptor protein extracellular domain positions 131-147 with additional Cys at the C-terminus and phosphoserine at position 137, KBP0049p peptide
HGVKFRSHQFTKIDTIAC

SEQ ID NO: 7 - protein, 18 aa, Artificial Sequence
  synthetic EphA3 receptor protein extracellular domain positions 131-147 with additional Cys at the C-terminus and Ser at position 137, KBP0049 peptide
HGVKFRSHQFTKIDTIAC

SEQ ID NO: 8 - protein, 18 aa, Artificial Sequence
  synthetic EphA3 receptor protein extracellular domain  positions 131-147 with additional Cys at the C-terminus and Glu at position 137, KBP0051 peptide
HGVKFREHQFTKIDTIAC

SEQ ID NO: 9 - protein, 18 aa, Artificial Sequence
  synthetic EphA3 receptor protein extracellular domain  positions 131-147 with additional Cys at the C-terminus and Asp at position 137, KBP0052 peptide
HGVKFRDHQFTKIDTIAC

SEQ ID NO: 10 - protein, 18 aa, Artificial Sequence
  synthetic EphA3 receptor protein extracellular domain  positions 131-147 with additional Cys at the C-terminus and Ala at position 137, KBP0053 peptide
HGVKFRAHQFTKIDTIAC

SEQ ID NO: 11 - protein, 100 aa, Artificial Sequence
  synthetic human germ-line VH3 3-73 heavy chain V-region
EVQLVESGGGLVQPGGSLKLSCAASGFTFSGSAMHWVRQASGKGLEWVGRIRSKANSYATAYAASVKGRFTISRDDSKNTAYLQMNSLKTEDTAVYYCTR

SEQ ID NO: 12 - protein, 120 aa, Artificial Sequence
  synthetic human engineered MI26-11E heavy chain V-region
EVQLVESGGGLVQPKGSLKLSCAASGFSIYTYALFWVRQAPGKGLEWVARIRSRSKNYATYYADSVKDRFTISRDDSKNMLYLQMNSLKTEDTAVYYCVLWSYSRALDYWGQGTLVTVSS

SEQ ID NO: 13 - protein, 120 aa, Artificial Sequence
  synthetic human engineered MI135-3F heavy chain V-region
EVQLVESGGGLVQPGGSLRLSCAASGFSIYTYALFWVRQAPGKGLEWVARIRSRSKSYATAYAASVKGRFTISRDDSKNMLYLQMNSLKTEDTAVYYCVLWSYSRALDYWGQGTLVTVSS

SEQ ID NO: 14 - protein, 120 aa, Artificial Sequence
  synthetic human engineered MI135-11G heavy chain V-region
EVQLVESGGGLVQPGGSLRLSCAASGFSIYTYALFWVRQAPGKGLEWVARIRSRSKNYATAYAASVKGRFTISRDDSKNMLYLQMNSLKTEDTAVYYCVLWSYSRALDYWGQGTLVTVSS

SEQ ID NO: 15 - protein, 120 aa, Artificial Sequence
  synthetic human engineered MI135-11H heavy chain V-region
EVQLVESGGGLVQPGGSLRLSCAASGFSIYTYALFWVRQAPGKGLEWVTRIRSKSKSYATAYAASVKGRFTISRDDSKNMLYLQMNSLKTEDTAVYYCVLWSYSRALDYWGQGTLVTVSS

SEQ ID NO: 16 - protein, 120 aa, Artificial Sequence
  synthetic human engineered MI136-7H heavy chain V-region
EVQLVESGGGVVQPGRSLRLSCAASGFTFYTSALYWVRQAPGKGLEWVARIRSRSKNYATYYADSVKDRFTISRDDSKNMLYLQMNSLKTEDTAVYYCVLWSYSRALDYWGQGTLVTVSS

SEQ ID NO: 17 - protein, 120 aa, Artificial Sequence
  synthetic human engineered MI137-2F heavy chain V-region
EVQLVESGGDLVKPGGALRLSCAASGFSIYTYALFWVRQAPGKGLEWVARIRSRSKNYATYYADSVKDRFTISRDDSKNMLYLQMNSLKTEDTAVYYCVLWSYSRALDYWGQGTLVTVSS

SEQ ID NO: 18 - protein, 120 aa, Artificial Sequence
  synthetic human engineered MI137-2G and MI137-11B heavy chain V-region
EVQLVESGGGVVQPGRSLRLSCAASGFSIYTYALFWVRQAPGKGLEWVARIRSRSKNYATYYADSVKDRFTISRDDSKNMLYLQMNSLKTEDTAVYYCVLWSYSRALDYWGQGTLVTVSS

SEQ ID NO: 19 - protein, 120 aa, Artificial Sequence
  synthetic human engineered MI137-6B heavy chain V-region
EVQLVESGGGLVKPGGSLRLSCAASGFSIYTYALFWVRQAPGKGLEWVARIRSRSKNYATYYADSVKDRFTISRDDSKNMLYLQMNSLKTEDTAVYYCVLWSYSRALDYWGQGTLVTVSS

SEQ ID NO: 20 - protein, 120 aa, Artificial Sequence
  synthetic human engineered MI137-9D heavy chain V-region
EVQLVESGGGLVQPGGSLRLSCAASGFSIYTYALFWVRQAPGKGLEWVARIRSRSKNYATAYADSVKDRFTISRDDSKNMLYLQMNSLKTEDTAVYYCVLWSYSRALDYWGQGTLVTVSS

SEQ ID NO: 21 - protein, 120 aa, Artificial Sequence
  synthetic human engineered MI137-9E heavy chain V-region
EVQLVESGGGLVQPGGSLRLSCAASGFSIYTYALFWVRQAPGKGLEWVARIRSRSKNYATYYADSVKDRFTISRDDSKNMLYLQMNSLKTEDTAVYYCVLWSYSRALDYWGQGTLVTVSS

SEQ ID NO: 22 - protein, 120 aa, Artificial Sequence; identical to SEQ ID NO: 21
  synthetic human engineered MI137-12D heavy chain V-region

SEQ ID NO: 23 - protein, 120 aa, Artificial Sequence
  synthetic human engineered optimized reference (opRef) heavy chain V-region
EVQLVESGGGLVQPKGSLKLSCAASGFSIYTYALFWVRQAPGKGLEWVARIRSRSKNYATYYADSVKDRFTVSRVDSRNMVYLQMTHLKTEDSAIYYCVLWSYSRALDYWGQGTLVTVSS

SEQ ID NO: 24 - protein, 95 aa, Artificial Sequence
  synthetic human germ-line VkI L18 light chain V-region
AIQLTQSPSSLSASVGDRVTITCRASQGISSALAWYQQKPGKAPKLLIYDASSLESGVPSRFSGSGSGTDFTLTISSLQPEDFATYYCQQFNSYP

SEQ ID NO: 25 - protein, 106 aa, Artificial Sequence
  synthetic human engineered MI119-2-2C light chain V-region
DIQMTQSPSSLSASVGDRVTITCRASSGISGIYWYQQKPGKAPKLLIYDASSLYTGVPSRFSGSGSGTEFTLTISSLQPEDFATYYCHQRSNYPYTFGQGTKLEIK

SEQ ID NO: 26 - protein, 106 aa, Artificial Sequence
  synthetic human engineered MI119-3-3A light chain V-region
DIQMTQSPSSLSASVGDRVTITCSASSSISDIYWYQQKPGKAPKLLIYDASNLYSGVPSRFSGSGSGTEFTLTISSLQPEDFATYYCHQRSNYPYTFGQGTKLEIK

SEQ ID NO: 27 - protein, 106 aa, Artificial Sequence
  synthetic human engineered MI121-10 light chain V-region
DIQMTQSPSSVSASVGDRVTITCSASQGIGAIYWYQQKPGKAPKLLIYDASNLDSGVPSRFSGSGSGTEFTLTISSLQPEDFATYYCHQRSNYPYTFGQGTKLEIK

SEQ ID NO: 28 - protein, 107 aa, Artificial Sequence
  synthetic human engineered MI121-A11 light chain V-region
DIQMTQSPSSLSASVGDRVTITCRASQSISNALYWYQQKPGKAPKLLIYDASSLYTGVPSRFSGSGSGTEFTLTISSLQPEDFATYYCHQRSNYPYTFGQGTKLEIK

SEQ ID NO: 29 - protein, 107 aa, Artificial Sequence
  synthetic human engineered MI121-B3 light chain V-region
DIQMTQSPSSLSASVGDRVTITCRASQSISSAIAWYQQKPGKAPKLLIYDASSLETGVPSRFSGSGSGTEFTLTISSLQPEDFATYYCHQRSNYPYTFGQGTKLEIK

SEQ ID NO: 30 - protein, 107 aa, Artificial Sequence
  synthetic human engineered MI121-D8 light chain V-region
DIQMTQSPSSLSASVGDRVTITCRASQSISSYIYWYQQKPGKAPKLLIYDTSNLYTGVPSRFSGSGSGTEFTLTISSLQPEDFATYYCHQRSNYPYTFGQGTKLEIK

SEQ ID NO: 31 - protein, 106 aa, Artificial Sequence
  synthetic human engineered MI126-B1 light chain V-region
DIQMTQSPSSLSASVGDRVTITCRASSGISDLYWYQQKPGKAPKLLIYDASKLESGVPSRFSGSGSGTEFTLTISSLQPEDFATYYCHQRSNYPYTFGQGTKLEIK

SEQ ID NO: 32 - protein, 106 aa, Artificial Sequence
  synthetic human engineered MI126-B9 light chain V-region
DIQMTQSPSSLSASIGGRVTITCRASSSISNISWYQQKPGKAPKLLIYDASKLQSGVPSRFSGSGSGTEFTLTISSLQPEDFATYYCHQRSNYPYTFGQGTKLEIK

SEQ ID NO: 33 - protein, 106 aa, Artificial Sequence
  synthetic human engineered MI126-C10 light chain V-region
DIQMTQSPSSLSASVGDRVTITCSASSGISNLAWYQQKPGKAPKLLIYDTSKLQTGVPSRFSGSGSGTEFTLTISSLQPEDFATYYCHQRSNYPYTFGQGTKLEIK

SEQ ID NO: 34 - protein, 106 aa, Artificial Sequence
  synthetic human engineered MI126-D2 light chain V-region
DIQMTQSPSSLSASVGDRVTITCRASSGVSDIYWYQQKPGKAPKLLIYDASKLESGVPSRFSGSGSGTEFTLTISSLQPEDFATYYCHQRSNYPYTFGQGTKLEIK

SEQ ID NO: 35 - protein, 107 aa, Artificial Sequence
  synthetic human engineered MI127-E1 light chain V-region
DIQMTQSPSSVSASVGDRVTITCRASQSISNYLYWYQQKPGKAPKLLIYDASKLQSGVPSRFSGSGSGTEFTLTISSLQPEDFATYYCHQRSNYPYTFGQGTKLEIK

SEQ ID NO: 36 - protein, 107 aa, Artificial Sequence
  synthetic human engineered MI127-E11 light chain V-region
DIQMTQSPSSLSASVGDRVTITCRASQGVSDYIYWYQQKPGKAPKLLIYDASKLQSGVPSRFSGSGSGTEFTLTISSLQPEDFATYYCHQRSNYPYTFGQGTKLEIK

SEQ ID NO: 37 - protein, 107 aa, Artificial Sequence
  synthetic human engineered MI127-F11 light chain V-region
DIQMTQSPSAMSASVGDRVTITCRASQSISNALAWYQQKPGKAPKLLIYDASKLETGVPSRFSGSGSGTEFTLTISSLQPEDFATYYCHQRSNYPYTFGQGTKLEIK

SEQ ID NO: 38 - protein, 107 aa, Artificial Sequence
  synthetic human engineered MI127-F7 light chain V-region
DIQMTQSPSPLSASVGDRVTITCRASQGISDDIYWYQQKPGKAPKLLIYDASKLESGVPSRFSGSGSGTEFTLTISSLQPEDFATYYCHQRSNYPYTFGQGTKLEIK

SEQ ID NO: 39 - protein, 106 aa, Artificial Sequence
  synthetic human engineered optimized reference (opRef) light chain V-region
EIVLTQSPAIMSASPGEKVTLTCSASSSISDIYWYQQKPGTSPKRWIYDTSKLSSGVPTRFSGSGSGTSYSLTISSLEAEDAATYYCHQRSNYPYTFGQGTKLEIK

SEQ ID NO: 40 - protein, 95 aa, Artificial Sequence
  synthetic human germ-line VkIII L6 light chain V-region
EIVLTQSPATLSLSPGERATLSCRASQSVSSYLAWYQQKPGQAPRLLIYDASNRATGIPARFSGSGSGTDFTLTISSLEPEDFAVYYCQQRSNWP

SEQ ID NO: 41 - protein, 106 aa, Artificial Sequence
  synthetic human engineered MI130-A1 light chain V-region
EIVLTQSPGTLSLSPGERATLSCRASSSISNIAWYQQKPGQAPRLLIYDTSSRSTGVPSRFSGSGSGTEFTLTISSLQPEDFATYYCHQRSNYPYTFGQGTKLEIK

SEQ ID NO: 42 - protein, 106 aa, Artificial Sequence
  synthetic human engineered MI130-B2 light chain V-region
EIVLTQSPGTLSLSPGERATLSCRASSSVSNLAWYQQKPGQAPRLLIYDTSNLYSGVPSRFSGSGSGTEFTLTISSLQPEDFATYYCHQRSNYPYTFGQGTKLEIK

SEQ ID NO: 43 - protein, 106 aa, Artificial Sequence
  synthetic human engineered MI130-D4 light chain V-region
EIVLTQSPGTLSLSPGERATLSCRASSSISSLAWYQQKPGQAPRLLIYDTSSRDSGVPSRFSGSGSGTEFTLTISSLQPEDFATYYCHQRSNYPYTFGQGTKLEIK

SEQ ID NO: 44 - protein, 107 aa, Artificial Sequence
  synthetic human engineered MI131-F2 light chain V-region
EIVLTQSPGTLSLSPGERATLSCRASQSISNYIYWYQQKPGQAPRLLIYDASSRYSGVPSRFSGSGSGTEFTLTISSLQPEDFATYYCHQRSNYPYTFGQGTKLEIK

SEQ ID NO: 45 - protein, 107 aa, Artificial Sequence
  synthetic human engineered MI131-F4 light chain V-region
EIVLTQSPGTLSLSPGERVTLSCRASQSISNYIYWYQQKPGQAPRLLIYDTSSLYSGVPSRFSGSGSGTEFTLTISSLQPEDFATYYCHQRSNYPYTFGQGTKLEIK

SEQ ID NO: 46 - protein, 107 aa, Artificial Sequence
  synthetic human engineered MI131-H9 light chain V-region
EIVLTQSPGTLSLSPGQRATLSCRASQSVSSALAWYQQKPGQAPRLLIYDASNRSSGVPSRFSGSGSGTEFTLTISSLQPEDFATYYCHQRSNYPYTFGQGTKLEIK

SEQ ID NO: 47 - protein, 106 aa, Artificial Sequence; identical to SEQ ID NO: 39
  synthetic human engineered optimized reference (opRef) light chain V-region

SEQ ID NO: 48 - protein, 106 aa, Mus sp.
  mouse anti-phosphoserine monoclonal antibody PSR-45 light chain V-region
EIVLTQSPAIMSASPGEKVTLTCSASSSISDIYWYQQKPGTSPKRWIYDTSKLSSGVPTRFSGSGSGTSYSLTISSLEAEDAATYYCHQRSNYPYTFGGGAKLEIK

SEQ ID NO: 49 - protein, 120 aa, Mus sp.
  mouse anti-phosphoserine monoclonal antibody PSR-45 heavy chain V-region
EVQLVESGGGLVQPKGSLKLSCAASGFSIYTYALFWVRQAPGKGLEWVARIRSRSKNYATYYADSVKDRFTVSRVDSRNMVYLQMTHLKTEDSAIYYCVLWSYSRALDYWGQGTSVTVSS

SEQ ID NO: 50 - protein, 6 aa, Artificial Sequence
  synthetic 6X His tag, H6
HHHHHH

SEQ ID NO: 51 - protein, 9 aa, Artificial Sequence
  synthetic mouse anti-phosphoserine monoclonal antibody PSR-45 heavy chain HCDR3
WSYSRALDY

SEQ ID NO: 52 - protein, 9 aa, Artificial Sequence
  synthetic human engineered anti-phosphoserine monoclonal antibody heavy chain MI20-F8-VH HCDR3
WSHSRARDY

SEQ ID NO: 53 - protein, 9 aa, Artificial Sequence
  synthetic human engineered anti-phosphoserine monoclonal antibody heavy chain MI20-C17-VH HCDR3
WSYSRFLDY

SEQ ID NO: 54 - protein, 9 aa, Artificial Sequence
  synthetic human engineered anti-phosphoserine monoclonal antibody heavy chain MI20-H22-VH HCDR3
WSHSRAMDY

SEQ ID NO: 55 - protein, 9 aa, Artificial Sequence
  synthetic human engineered anti-phosphoserine monoclonal antibody heavy chain MI20-H23-VH HCDR3
WSHSRALDY

SEQ ID NO: 56 - protein, 9 aa, Artificial Sequence
  synthetic human engineered anti-phosphoserine monoclonal antibody heavy chain MI20-B30-VH HCDR3
WSYSRSLDY

SEQ ID NO: 57 - protein, 9 aa, Artificial Sequence
  synthetic human engineered anti-phosphoserine monoclonal antibody heavy chain MI20-C32-VH HCDR3
WSYSRNLDY

SEQ ID NO: 58 - protein, 9 aa, Artificial Sequence
  synthetic human engineered anti-phosphoserine monoclonal antibody heavy chain MI20-G38-VH HCDR3
WSQSRALDY

SEQ ID NO: 59 - protein, 9 aa, Artificial Sequence
  synthetic human engineered anti-phosphoserine monoclonal antibody heavy chain MI20-D45-VH HCDR3
WSYSRGLHY

SEQ ID NO: 60 - protein, 9 aa, Artificial Sequence
  synthetic human engineered anti-phosphoserine monoclonal antibody heavy chain MI20-B48-VH HCDR3
WSYSRAADY

SEQ ID NO: 61 - protein, 9 aa, Artificial Sequence
  synthetic mouse anti-phosphoserine monoclonal antibody PSR-45 light chain LCDR3
HQRSNYPYT

SEQ ID NO: 62 - protein, 9 aa, Artificial Sequence
  synthetic human engineered anti-phosphoserine monoclonal antibody heavy chain MI21-B7-VK LCDR3
HQRSVYPYT

SEQ ID NO: 63 - protein, 9 aa, Artificial Sequence
  synthetic human engineered anti-phosphoserine monoclonal antibody heavy chain MI21-B10-VK LCDR3
HQRVNYPYT

SEQ ID NO: 64 - protein, 9 aa, Artificial Sequence
  synthetic human engineered anti-phosphoserine monoclonal antibody heavy chain MI21-D7-VK LCDR3
HQRSGYPYT

SEQ ID NO: 65 - protein, 9 aa, Artificial Sequence
  synthetic human engineered anti-phosphoserine monoclonal antibody heavy chain MI21-F6-VK LCDR3
HQRSNVPYT

SEQ ID NO: 66 - protein, 9 aa, Artificial Sequence
  synthetic human engineered anti-phosphoserine monoclonal antibody heavy chain MI21-F10-VK LCDR3
HQRSFYPYT

SEQ ID NO: 67 - protein, 9 aa, Artificial Sequence
  synthetic human engineered anti-phosphoserine monoclonal antibody heavy chain MI21-G7-VK LCDR3
HQRTNYPYT